(19) United States
(12) Patent Application Publication
(10) Pub. No.: US 2026/0310079 A1
(43) Pub. Date: Oct. 8, 2026

(54) LITHOGRAPHY METHOD

(71) Applicant: TAIWAN SEMICONDUCTOR MANUFACTURING COMPANY, LTD., Hsinchu (TW)

(72) Inventors: Yen-Hao CHEN, New Taipei City (TW); Wei-Han LAI, New Taipei City (TW); Kuan-Hsin LO, Nantou County (TW); Yahru CHENG, Taipei City (TW)

(73) Assignee: TAIWAN SEMICONDUCTOR MANUFACTURING COMPANY, LTD., Hsinchu (TW)

(21) Appl. No.: 19/091,168

(22) Filed: Mar. 26, 2025

(51) Int. Cl. G03F 7/09; C07C 31/20; C07C 31/22; C07C 39/08; C07F 5/02; C08B 37/16; G03F 7/16

(52) U.S. Cl. CPC G03F 7/094; C07C 31/20; C07C 31/202; C07C 31/225; C07C 39/08; C07F 5/025; C08B 37/0012; G03F 7/168

(57) ABSTRACT

A lithography method comprises the following steps. An auxiliary composition is applied over the target layer to form an auxiliary layer, wherein the auxiliary composition comprises a first polymer, the first polymer comprises a first functional group including a first boronic acid (BOH₂) group, a first multi-OH group, or a combination thereof. A photoresist composition is applied over the auxiliary layer to form a photoresist layer. The photoresist layer is exposed. The photoresist layer is developed.

### BACKGROUND

Semiconductor devices are used in a variety of electronic applications, such as, for example, personal computers, cell phones, digital cameras, and other electronic equipment. Semiconductor devices are typically fabricated by sequentially depositing insulating or dielectric layers, conductive layers, and semiconductor layers of material over a semiconductor substrate, and patterning the various material layers using lithography to form circuit components and elements thereon.

The semiconductor industry continues to improve the integration density of various electronic components (e.g., transistors, diodes, resistors, capacitors, etc.) by continual reductions in minimum feature size, which allow more components to be integrated into a given area. However, as the minimum features sizes are reduced, additional problems arise that should be addressed.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

FIG. 1 is a flowchart of an exemplary method for patterning a target layer in accordance with some embodiments.

FIGS. 2A, 4A, 5A, 6A, 7A, 8A, 9 and 10 are cross-sectional views of a semiconductor device at various stages of the method of FIG. 1 in accordance with various aspects of the present disclosure.

FIGS. 3A and 3B show the formula (a1) and the formula (a2) of the first polymer in accordance with some embodiments.

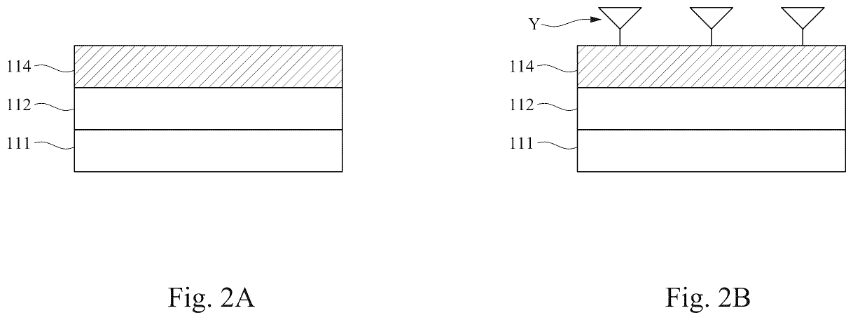

FIGS. 2B, 4B, 5B, 6B, 7B and 8B are exemplary chemical bonds between a photoresist layer and an auxiliary layer in FIGS. 2A, 4A, 5A, 6A, 7A and 8A, respectively, in accordance with some embodiments.

FIG. 11 illustrates an example of a Fin Field-Effect Transistors (FinFETs) in a three-dimensional view, in accordance with some embodiments.

FIGS. 12A, 12B, 13, 14, 15A, 15B, 16, 17A, 17B, 18A, 18B, 19A, 19B, 19C, 19D, 20A, 20B, 21A, 21B, 22A, 22B, 23A, 23B and 23C are cross-sectional views of intermediate stages in the manufacturing of FinFETs, in accordance with some embodiments.

### DETAILED DESCRIPTION

The following disclosure provides many different embodiments, or examples, for implementing different features of the invention. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Further, spatially relative terms, such as “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

In lithography methods, auxiliary layers may be formed under the photoresist layers to assist exposure and focus of the photoresist layers during patterning of the photoresist layers. However, the photoresist layers and the auxiliary layers have insufficient adhesion. Collapse or peeling of the photoresist layers may occur due to the tightened pitch and shrink of critical dimension (CD) in the advanced integrated circuit (IC) fabrication technology node.

Therefore, there is a need to reduce a thickness of the photoresist layer and adopt a footing profile for the photoresist layer to improve the peeling widow. However, thinner photoresist layers may cause insufficient etch budget, and the footing profile may increase the risk of scum defects after the etching process. Increasing the interaction force between the photoresist layer and the underlying auxiliary layer by performing surface treatment or modification to the auxiliary layer to increase the adhesion may not be strong enough because such interaction force is physical force.

Some embodiments of the present disclosure provide a novel auxiliary layer and a novel photoresist layer over the novel auxiliary layer in which the novel photoresist layer and the novel auxiliary layer could form a strong chemical bond therebetween to increase adhesion to each other. Therefore, collapse or peeling of the photoresist layer can be prevented. Peeling window of the photoresist layer can be improved, thus improving lithography performance.

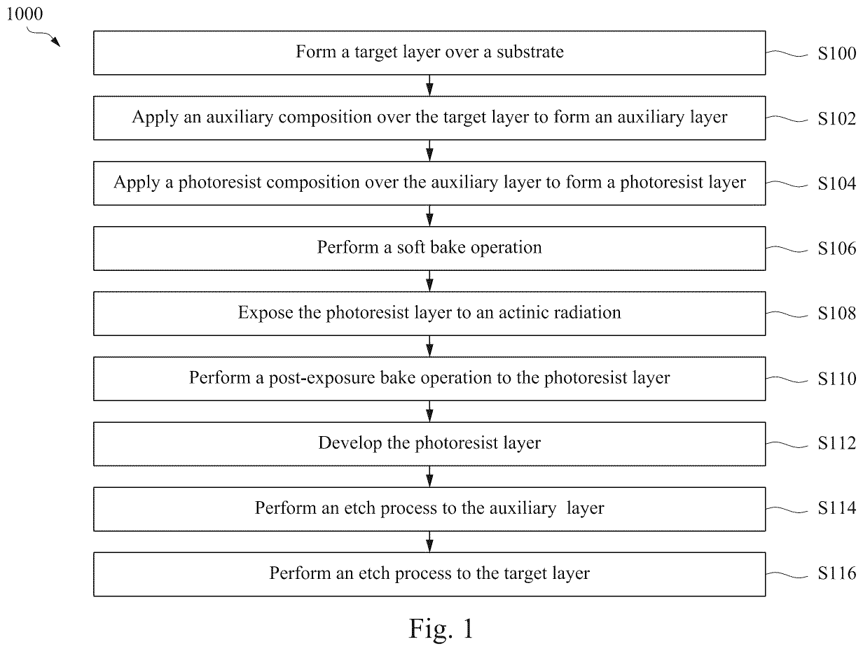

FIG. 1 is a flowchart of an exemplary method 1000 for patterning a target layer in accordance with some embodiments. FIGS. 2A, 4A, 5A, 6A, 7A, 8A, 9 and 10 are cross-sectional views of a semiconductor device at various stages of the method 1000 of FIG. 1 in accordance with various aspects of the present disclosure. FIGS. 2B, 4B, 5B, 6B, 7B and 8B are exemplary chemical bonds between a photoresist layer and an auxiliary layer in FIGS. 2A, 4A, 5A, 6A, 7A and 8A, respectively, in accordance with some embodiments.

The method 1000 includes a relevant part of an entire manufacturing process. It is understood that additional operations may be provided before, during and after the operations shown by FIG. 1, and some of the operations described below can be replaced or eliminated for additional embodiments of the method 1000. The order of the operations/processes may be interchangeable. The method 1000 includes fabrication of a semiconductor device. However, the fabrication of the semiconductor device is merely an example for describing the manufacturing process according to some embodiments of the present disclosure.

The method 1000 begins at operation S100 in which the operation S100 includes forming a target layer over a substrate. With reference to FIGS. 1 and 2A, in some embodiments of the operation S100, a target layer 112 to be patterned is formed over a substrate 111. For example, the target layer 112 may be formed by an acceptable deposition process, such as chemical vapor deposition (CVD), physical vapor deposition (PVD), atomic layer deposition (ALD), spin coating process, or the like. The substrate 111 may include an integrated circuit (IC) chip, system on chip (SoC), or portion thereof, and may include various passive and active microelectronic devices such as resistors, capacitors, inductors, diodes, metal-oxide semiconductor field effect transistors (MOSFET), complementary metal-oxide semiconductor (CMOS) transistors, bipolar junction transistors (BJT), laterally diffused MOS (LDMOS) transistors, high power MOS transistors, or other types of transistor.

In some embodiments, the substrate 111 is a silicon substrate doped with a p-type dopant such as boron (for example a p-type substrate). Alternatively, the substrate 111 could be another suitable semiconductor material. For example, the substrate 111 may be a silicon substrate that is doped with an n-type dopant such as phosphorous or arsenic (an n-type substrate). The substrate 111 could include other elementary semiconductors such as germanium and diamond. The substrate 111 could optionally include a compound semiconductor and/or an alloy semiconductor. Further, the substrate 111 could include an epitaxial layer (epi layer), may be strained for performance enhancement, and may include a silicon-on-insulator (SOI) structure.

In some embodiments, the target layer 112 is substantially conductive or semi-conductive. The electrical resistance may be less than about 103 ohm-meter. In some embodiments, the target layer 112 contains metal, metal alloy, or metal nitride/sulfide/selenide/oxide/silicide with the formula MXₐ, where M is a metal, and X is N, S, Se, O, Si, and where “a” is in a range from about 0.4 to 2.5. For example, the target layer 112 may contain Ti, Al, Co, Ru, TiN, WN₂, or TaN.

In some other embodiments, the target layer 112 contains a dielectric material with a dielectric constant in a range from about 1 to about 40. In some other embodiments, the target layer 112 contains Si, metal oxide, or metal nitride, where the formula is MX_{b}, wherein M is a metal or Si, and X is N or O, and wherein “b” is in a range from about 0.4 to 2.5. For example, the target layer 112 may contain SiO₂, silicon nitride, aluminum oxide, hafnium oxide, or lanthanum oxide.

The method 1000 then proceeds to operation S102 in which the operation S102 includes applying an auxiliary composition over the target layer to form an auxiliary layer. Still with reference to FIGS. 1, 2A and 2B, in some embodiments of the operation S102, an auxiliary composition is applied on the target layer 112 to form an auxiliary layer 114. The auxiliary composition applied on the target layer 112 to form the auxiliary layer 114 may be applied by spin coating process or deposition process.

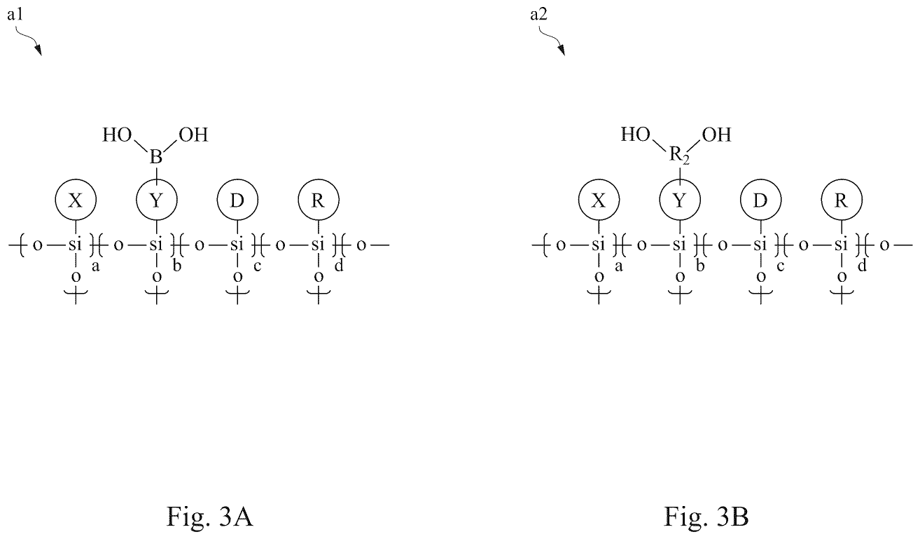

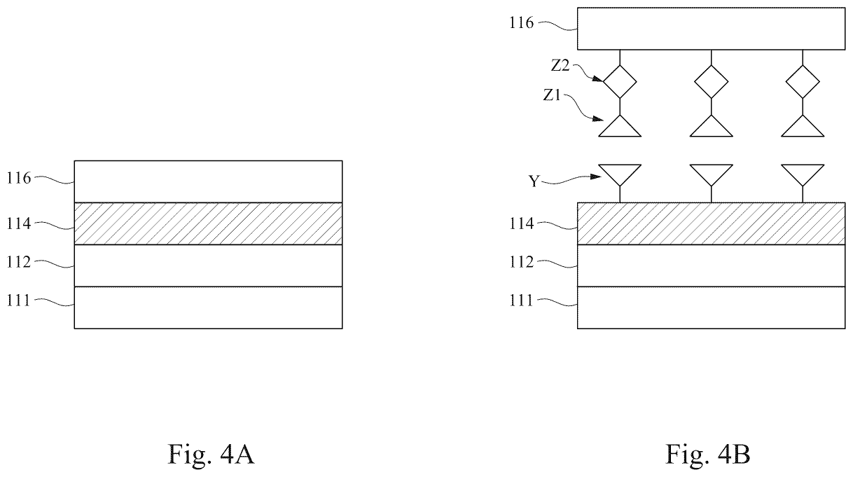

In some embodiments, the auxiliary composition has a first polymer, a cross linker, an additive and a solvent. In some embodiments, the first polymer can include a formula (a1) and a formula (a2). FIGS. 3A and 3B show the formula (a1) and the formula (a2) of the first polymer in accordance with some embodiments. Reference is made to FIGS. 3A and 3B. In some embodiments, the first polymer may include a first functional group “Y”, a second functional group “X”, a third functional group “D” and a fourth functional group “R” different from each other. For example, the first functional group “Y” can include a boronic acid group (BOH₂) (see FIG. 3A) or a multi-OH group (R₂(OH)₂) (see FIG. 3B) which functions as crosslinking sites to undergo crosslink reaction with a subsequently formed overlying photoresist layer (see FIGS. 4A-4B). In some embodiments, the second functional group “X” can include organic groups containing crosslinking sites as well. For example, the second functional group “X” can be double bonds, triple bonds, or the like. In some embodiments, the third functional group “D” can include aromatic structures or organic dyes. In some embodiments, the fourth functional group “R” can include organic groups used to provide adhesion with the photoresist layer layer (see FIGS. 4A-4B). In some embodiments, in the formula (a1) and the formula (a2) of the first polymer in FIGS. 3A and 3B, a+b+c+d=1. In some embodiments, the first functional group “Y” may be a t-butyl acetate group, an O-nitrobenzyl group, a pyrene group, the like, or a combination thereof. In some embodiments, the t-butyl acetate group can include a formula (a3), the O-nitrobenzyl group can include a formula (a4), and the pyrene group can include a formula (a5):

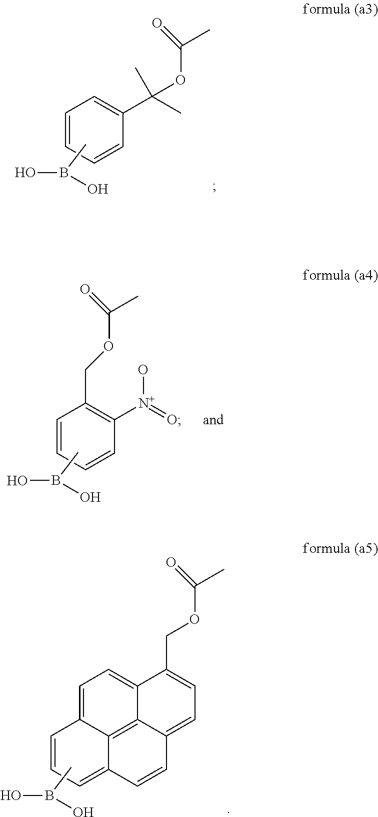

In some embodiments where the first functional group “Y” including the multi-OH group (R₂(OH)₂), the R₂(OH)₂ can be formulae (b1)-(b5):

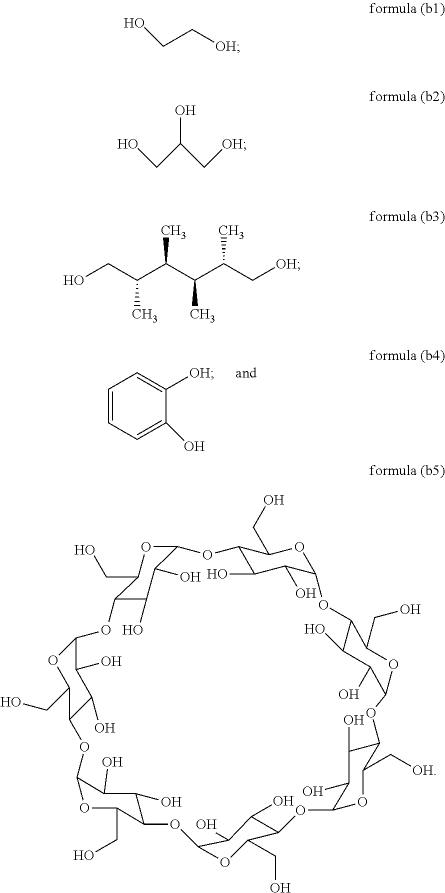

The method 1000 then proceeds to operation S104 in which the operation S104 includes applying a photoresist composition over the auxiliary layer to form a photoresist layer. With reference to FIGS. 1, 4A and 4B, in some embodiments of the operation S104, a photoresist composition is applied on the auxiliary layer 114 to form a photoresist layer 116. The photoresist composition applied on the auxiliary layer 114 to form the photoresist layer 116 may be applied by spin coating process or deposition process. The auxiliary layer 114 and the photoresist layer 116 can be collectively referred to as a multilayer stack used for lithography process.

In some embodiments, the photoresist composition has a second polymer, a photoacid generator (PAG), a quencher, and a solvent. The photoresist composition can be exposed by exposure tools using actinic radiation including at least one of the KrF, ArF, extreme ultraviolet (EUV) radiation, E-beam or the like. In some embodiments, the second polymer may include a fifth functional group “Z1”. For example, the fifth functional group “Z1” may be a t-butyl acetate group, an O-nitrobenzyl group, a pyrene group, the like, or a combination thereof. The fifth functional group “Z1” is configured to crosslink with, for example, the first functional group “Y” of the auxiliary layer 114, which would be discussed in greater detail below. The second polymer may include an acid labile group (ALG) “Z2” or acid leaving group. The photoacid generator is configured to produce an acid upon a radiation. The acid cleaves the ALG “Z2” off the second polymer in a chemical amplification reaction. In some embodiments, the second polymer may include a formula (c1) or a formula (c2).

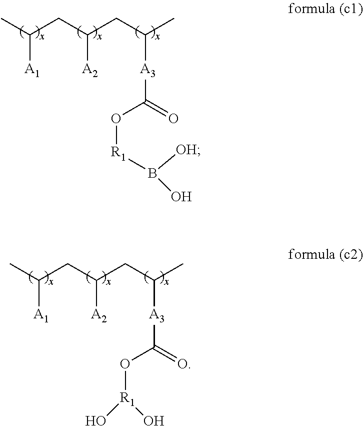

In the formulae (c1) and (c2), A₁, A₂, A₃ can be a C₁₋₃₀ benzyl group, a C₁₋₃₀ alkyl group, a C₁₋₃₀ cycloalkyl group, a C₁₋₃₀ hydroxyl alkyl group, a C₁₋₃₀ alkoxy group, a C₁₋₃₀ alkoxyl alkyl group, a C₁₋₃₀ acetyl group, a C₁₋₃₀ acetyl alkyl group, carboxyl group, a C₁₋₃₀ alky carboxyl group, a C₁₋₃₀ cycloalkyl carboxyl group, a C₁₋₃₀ saturated or unsaturated hydrocarbon ring or a C₁₋₃₀ heterocyclic group which can be a chain, a ring, 3D structure, such as an adamantyl group. In the formulae (c1) and (c2), R₁ can be an acid leaving group and be bonded to a boronic acid, forming an R₁BOH₂ group or bonded to a multi-OH group, forming an R₁(OH)₂ group, in which R₁ is a C₄₋₁₅ alkyl group, a C₄₋₁₅ cycloalkyl group, a C₄₋₁₅ hydroxyl alkyl group, a C₄₋₁₅ alkoxy group, a C₄₋₁₅ alkoxyl alkyl group with a structure including a long chain, a cyclic structure, or a 3D structure. In the formulae (c1) and (c2), 0≤x≤1, 0≤y≤1, 0≤z≤1. For example, the second polymer may include a formula (c3):

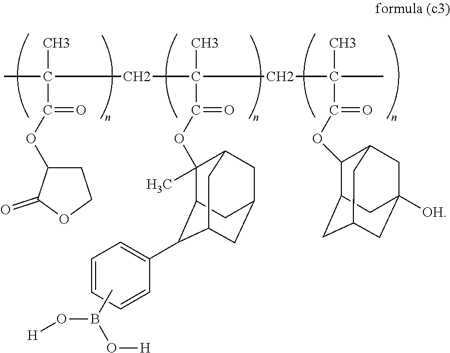

The method 1000 then proceeds to operation S106 in which the operation S106 includes performing a soft bake operation to the photoresist layer and the auxiliary layer. Still with reference to FIGS. 1 and 4A-4B, in some embodiments of the operation S106, a soft bake (SB) operation is performed to the photoresist layer 116 and the auxiliary layer 114 to reduce or remove the solvent in the photoresist layer 116 and the auxiliary layer 114 and promote crosslink reaction between the photoresist layer 116 and the auxiliary layer 114. For example, the soft bake operation promotes crosslink reaction between the first functional group “Y” of the first polymer of the auxiliary layer 114 and the fifth functional group “Z1” of the second polymer of the photoresist layer 116 in which the crosslink reaction can be represented by a chemical reaction (d1):

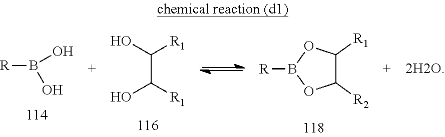

Product 118 and water (H₂O) can be reaction products of the auxiliary layer 114 and the photoresist layer 116. As shown in the product 118, a chemical bond can be formed and thus increase adhesion between the auxiliary layer 114 and the photoresist layer 116, and hence collapse or peeling of the photoresist layer 116 can be prevented.

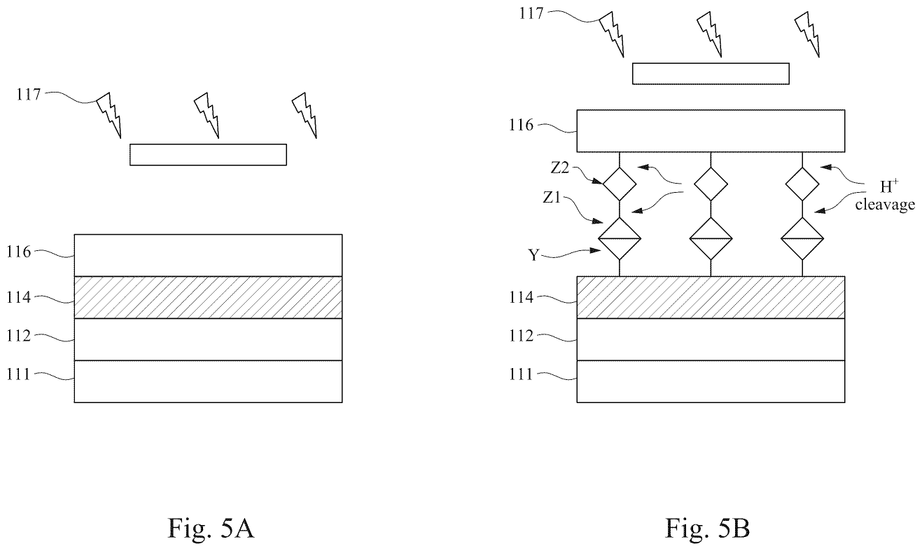

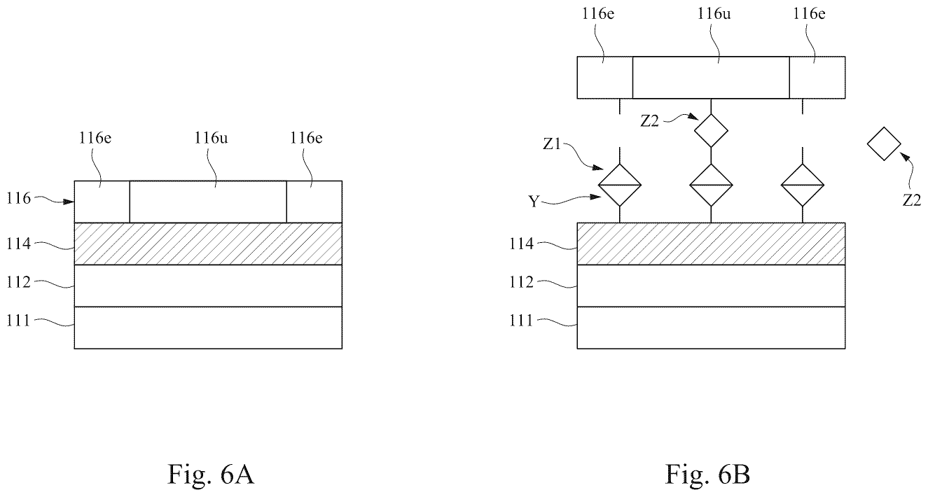

The method 1000 proceeds to operation S108 in which the operation S108 includes exposing the photoresist layer to an actinic radiation. With reference to FIGS. 1 and 5A-5B, in some embodiments of the operation S108, the photoresist layer 116 is exposed to an actinic radiation 117. For example, the actinic radiation 117 may include at least one of the KrF, ArF, extreme ultraviolet (EUV) radiation, E-beam or the like. The photoresist layer 116 may include an exposed region 116e and an unexposed region 116u (see FIGS. 6A-6B). In the present embodiment, the exposed region 116e can undergo some chemical changes in response to the actinic radiation 117. For example, with respect to FIG. 5B, the PAG (not shown) releases an acid (H⁺) in response to the actinic radiation 117. The acid cleaves the ALG “Z2” off the polymer backbone of the second polymer, which makes the second polymer more hydrophilic. As a result, the exposed region of the photoresist layer 116 becomes soluble in an aqueous developer, such as tetramethyl ammonium hydroxide (TMAH).

The method 1000 proceeds to operation S110 in which the operation S110 includes performing a post-exposure bake (PEB) operation to the photoresist layer. With reference to FIGS. 1, 6A and 6B, in some embodiments of the operation S110, a post-exposure bake operation is performed to the photoresist layer 116. The PEB operation can accelerates the above acid-generation and cleaving reactions. The ALG “Z2” of exposed region 116e are cleaved by the acids and the exposed region becomes more hydrophilic.

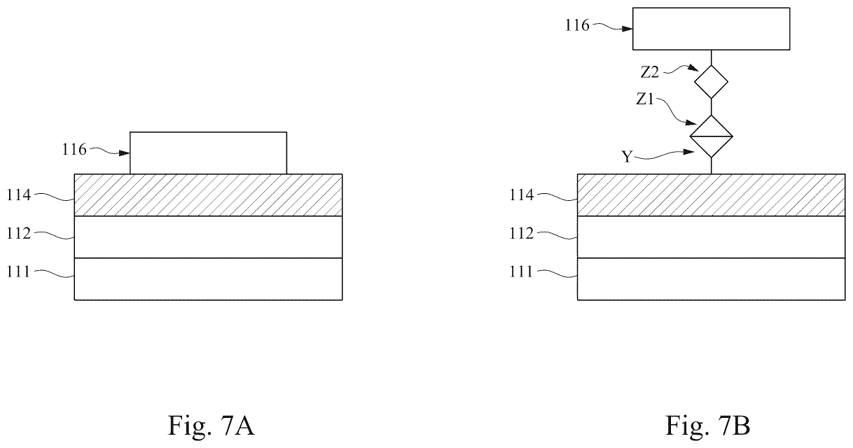

The method 1000 proceeds to operation S112 in which the operation S112 includes developing the photoresist layer. With reference to FIGS. 1 and 7A-7B, in some embodiments of the operation S112, the photoresist layer 116 is subsequently developed by applying a developer to the photoresist layer 116. In some embodiments, the developer is an aqueous solvent such as having 2.38% tetramethyl ammonium hydroxide (TMAH). The exposed region 116e of the photoresist layer 116 may be removed by the developer while the unexposed region 116u of the photoresist layer 116 remains.

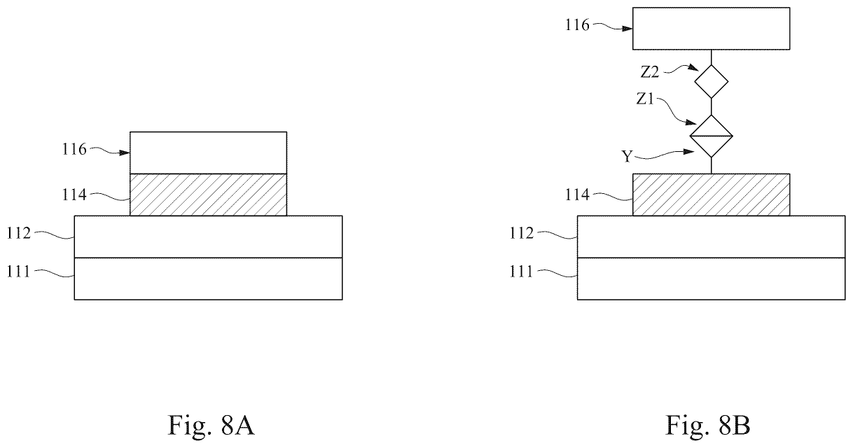

The method 1000 proceeds to operation S114 in which the operation S114 includes performing an etch process to the auxiliary layer. With reference to FIGS. 1 and 8A-8B, in some embodiments of the operation S114, an etch process is performed to the auxiliary layer 114 using the unexposed region of the photoresist layer 116 as an etch mask. For example, the etch process is a dry etch process including a biased plasma etch process that uses a chlorine-based chemistry, CF₄, NF₃, SF₆, or the like. The dry etch process may be performed anisotropically. As discussed previously with regard to FIGS. 4A-4B, since the photoresist layer 116 and the auxiliary layer 114 form the strong chemical bond therebetween to increase adhesion to each other, collapse or peeling of the photoresist layer 116 can be prevented. Peeling window of the photoresist layer 116 can be improved, thus improving lithography performance. The exposed region of the photoresist layer 116 can be removed after etching the auxiliary layer 114 by using a suitable photoresist stripper solvent or by a photoresist ashing operation.

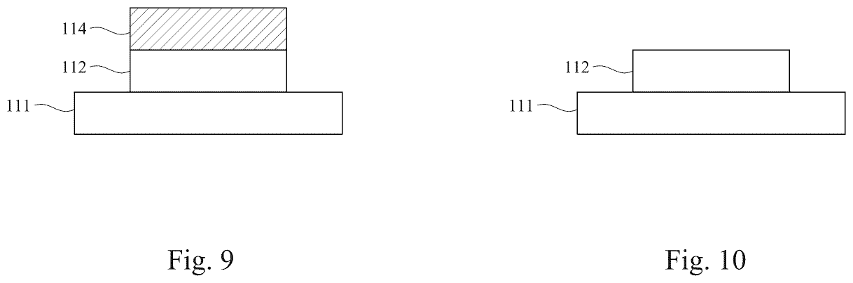

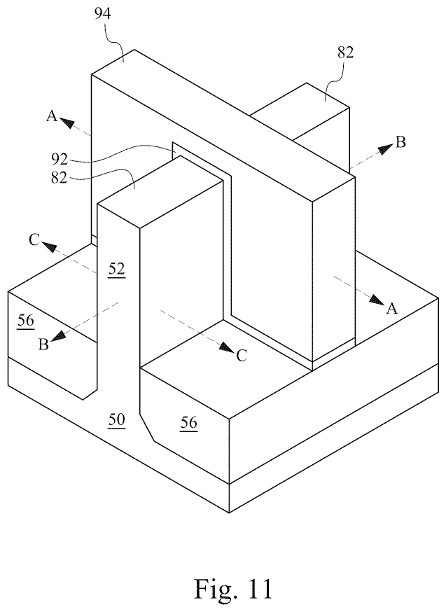

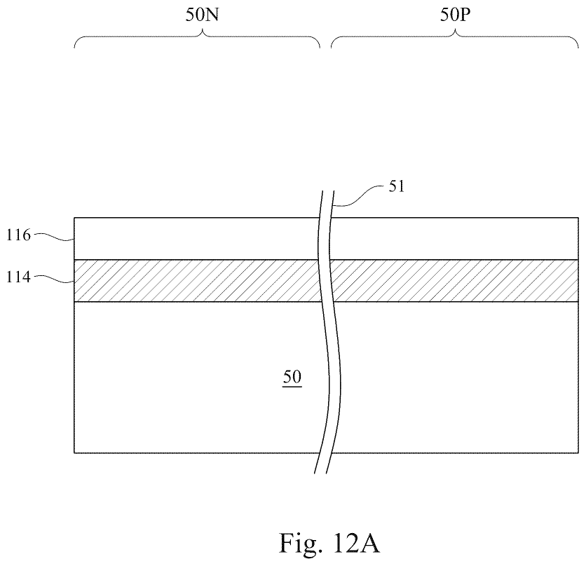

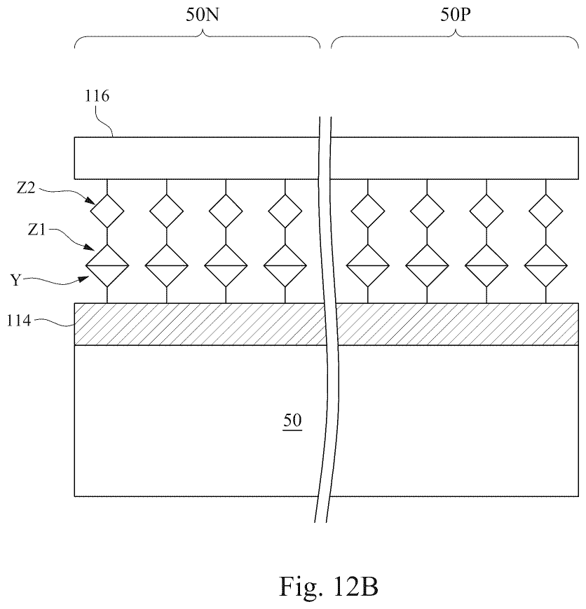

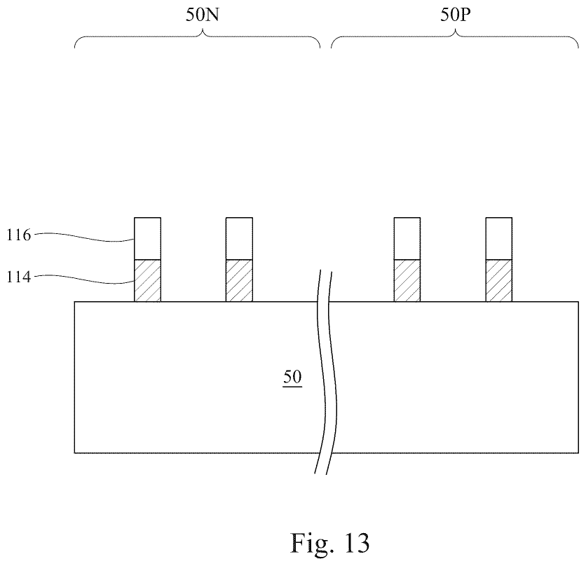

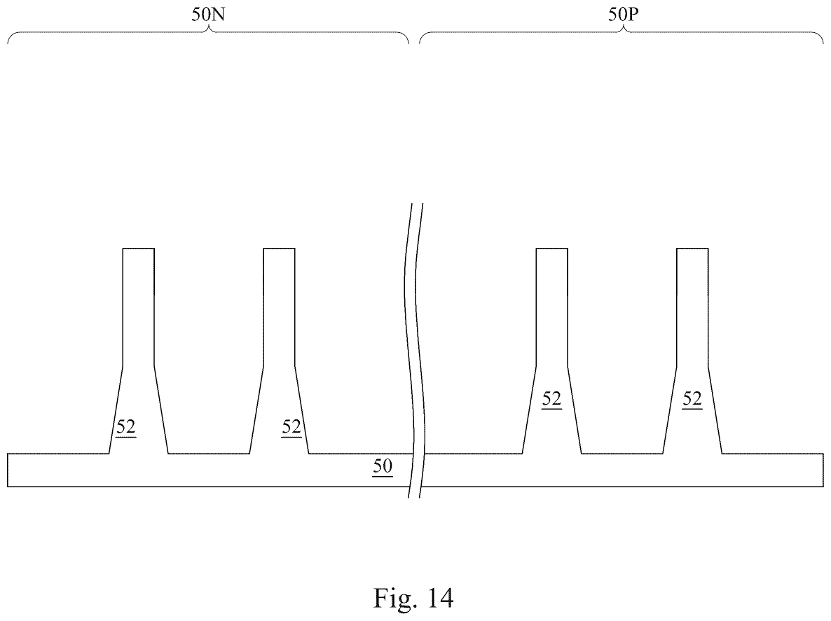

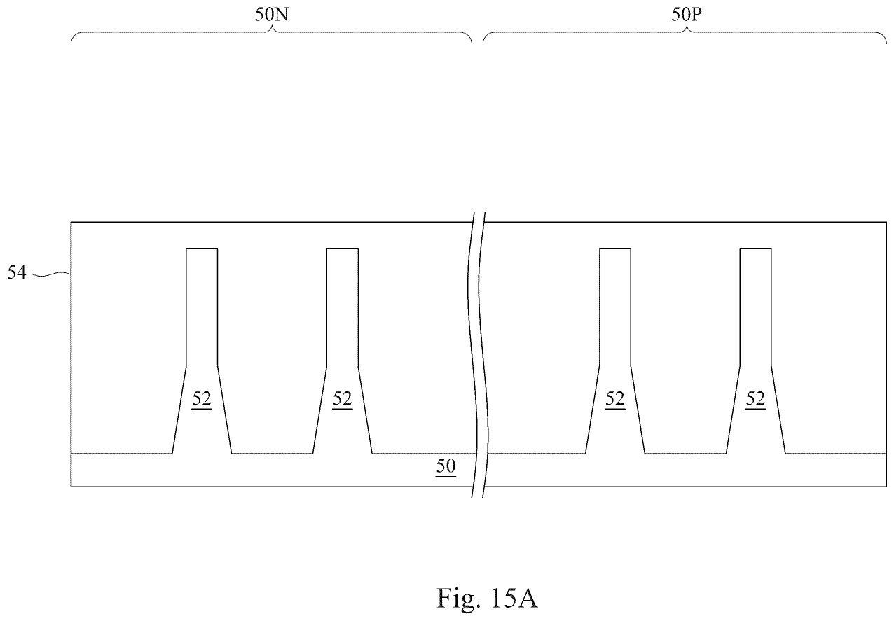

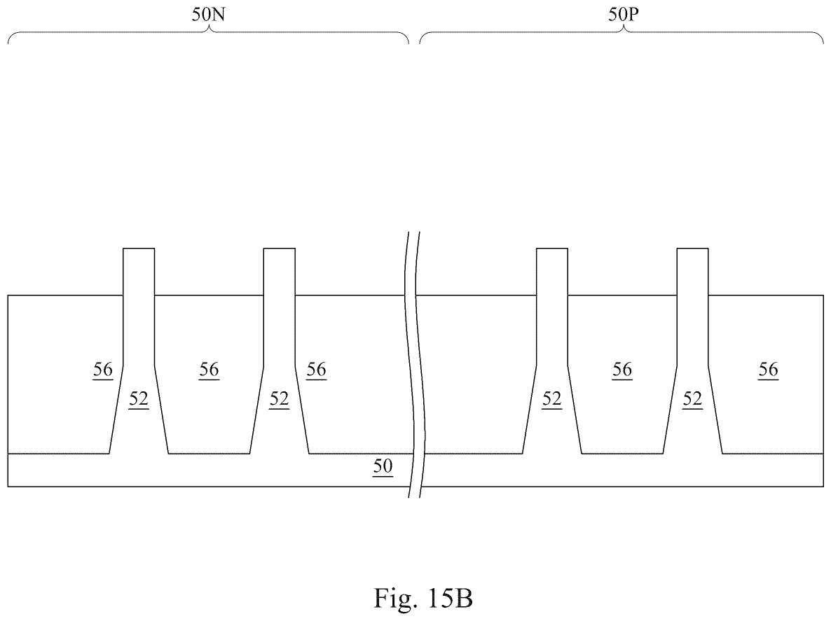

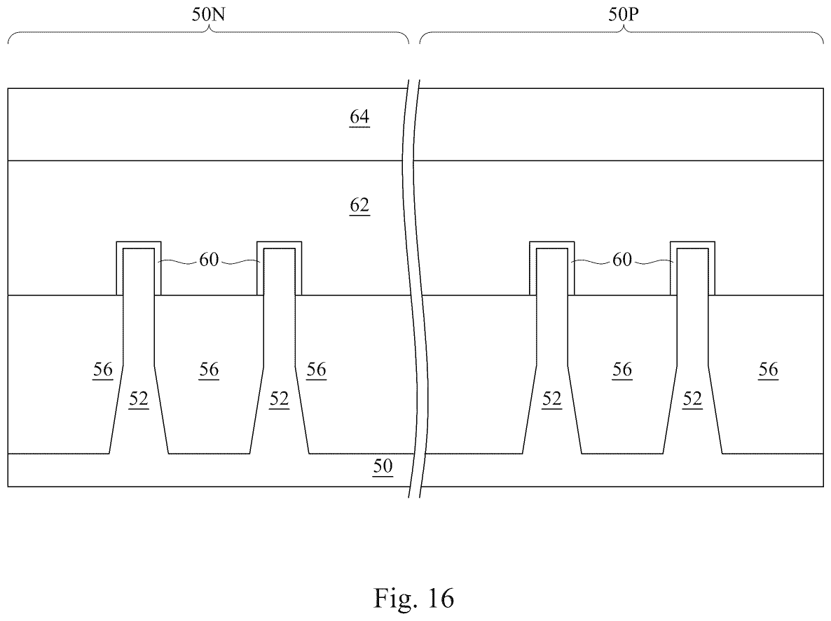

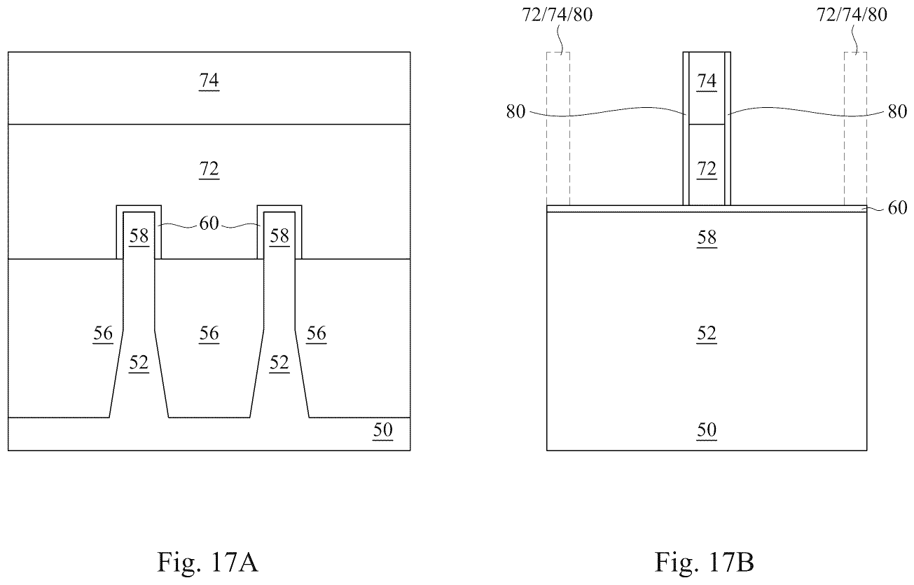

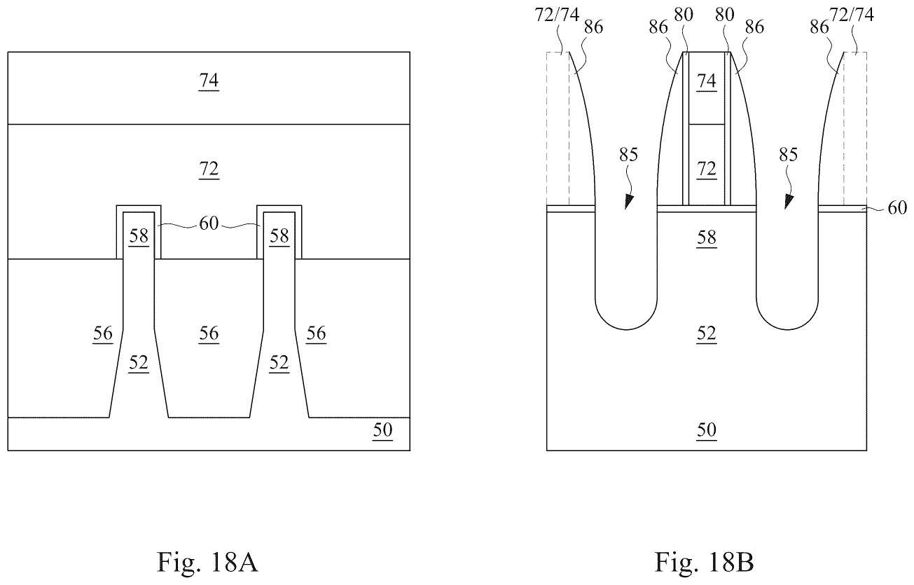

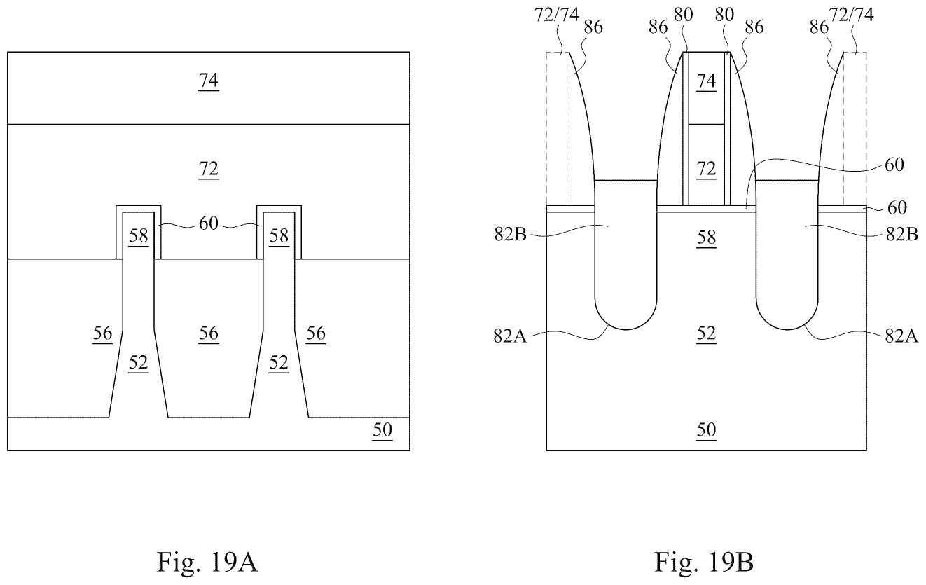

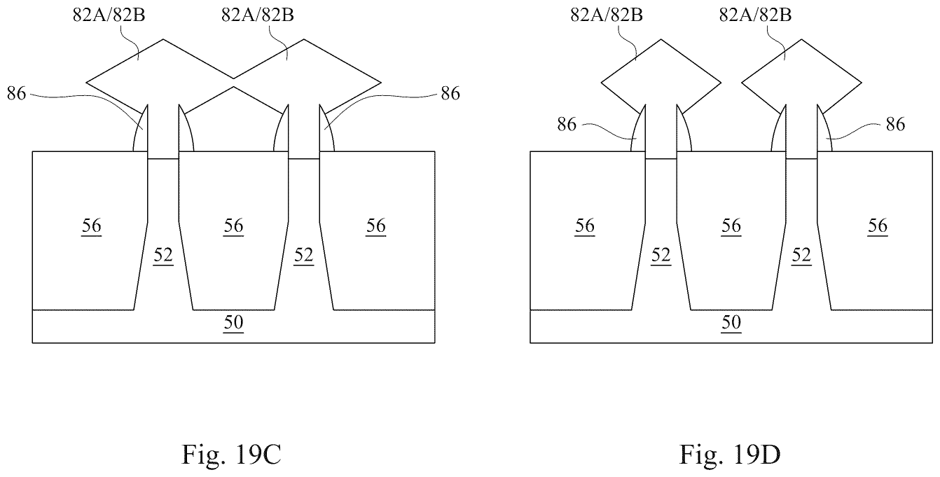

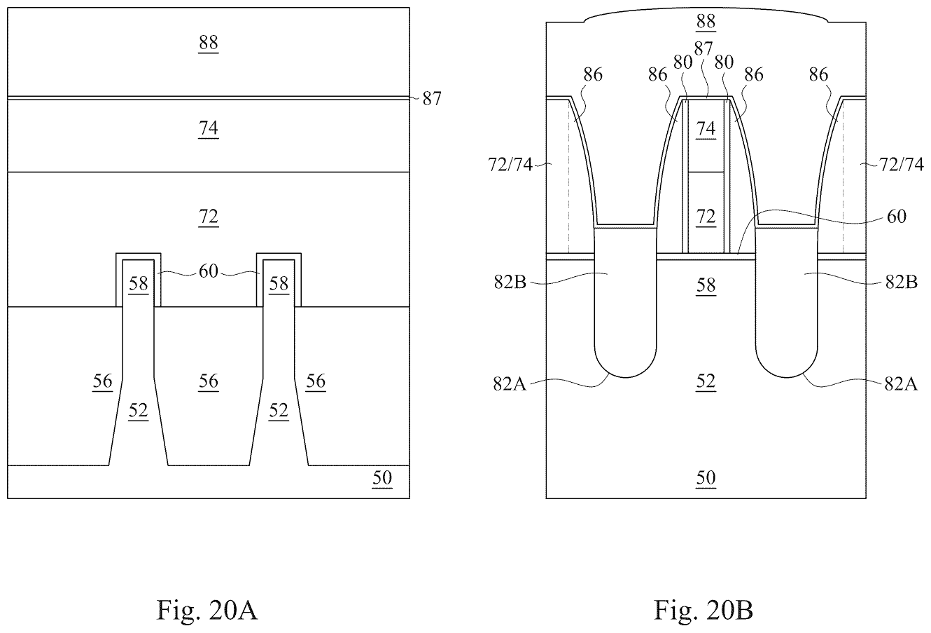

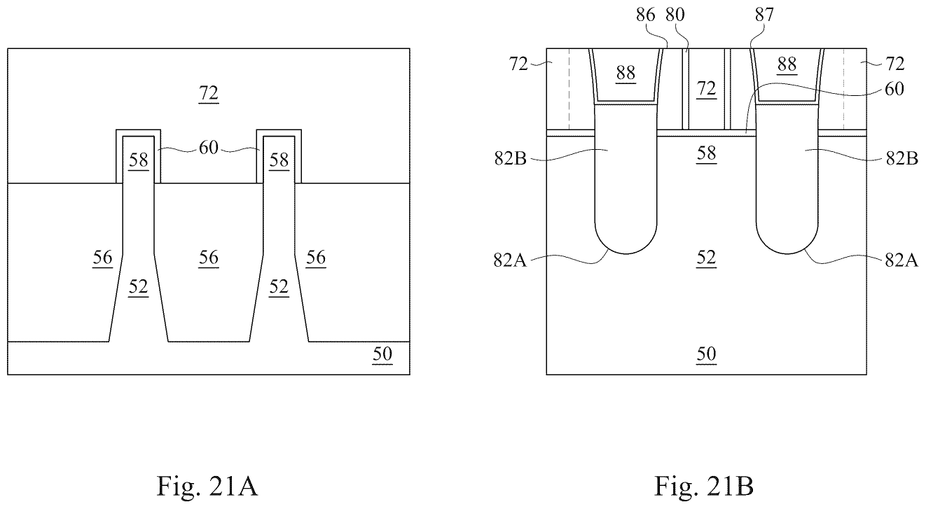

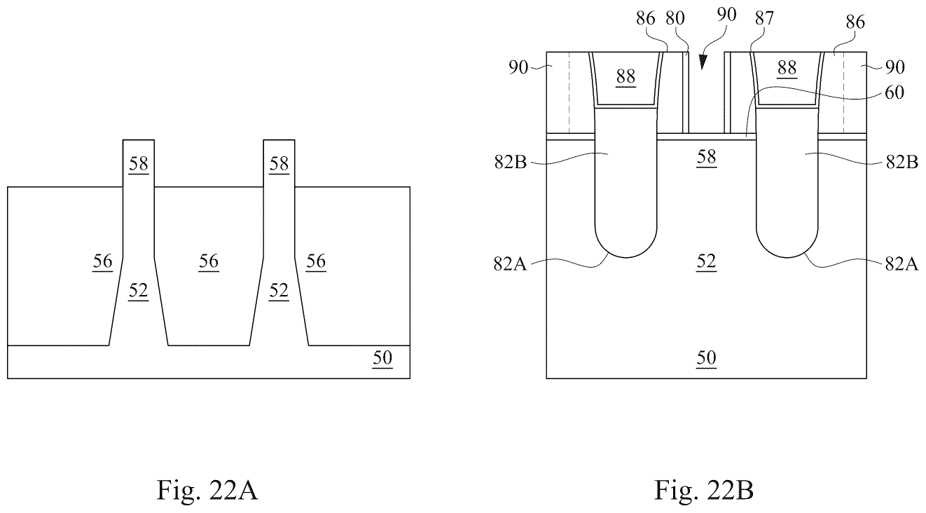

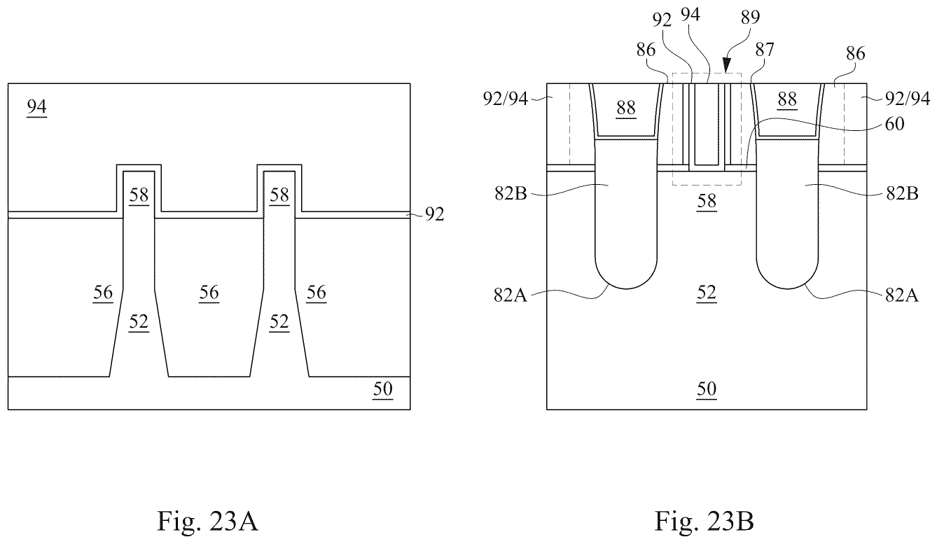

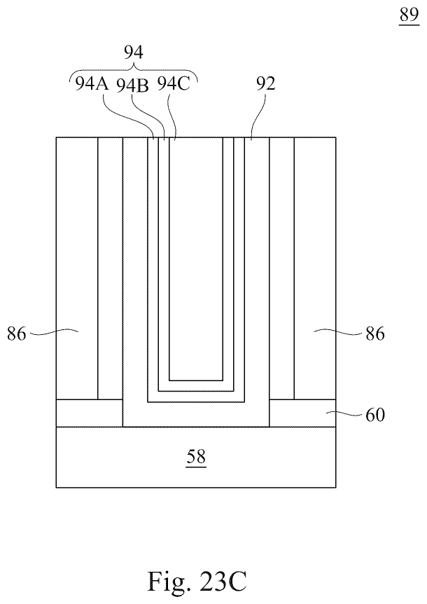

The method 1000 proceeds to operation S116 in which the operation S116 includes performing an etch process to the target layer. With reference to FIGS. 1 and 9, in some embodiments of the operation S116, an etch process is performed to the target layer 112 using the auxiliary layer 114 as an etch mask. For example, the etch process is a dry etch process including a biased plasma etch process that uses a chlorine-based chemistry, CF₄, NF₃, SF₆, or the like. The dry etch process may be performed anisotropically. The auxiliary layer 114 can be removed after etching the target layer 112. The resulting structure is shown in FIG. 10.

Fin Field-Effect Transistors (FinFETs) and methods of forming the same are provided in accordance with various embodiments. Intermediate stages of forming FinFETs are illustrated. Some embodiments discussed herein are discussed in the context of FinFETs formed using a gate-last (sometimes referred to as replacement gate process) process. In other embodiments, a gate-first process may be used. Some variations of the embodiments are discussed. Also, some embodiments contemplate aspects used in planar devices, such as planar FETs. One of ordinary skill in the art will readily understand other modifications that may be made that are contemplated within the scope of other embodiments. Although method embodiments are discussed in a particular order, various other method embodiments may be performed in any logical order and may include fewer or more steps described herein.

Before addressing the illustrated embodiments specifically, certain advantageous features and aspects of the present disclosed embodiments will be addressed generally. In general terms, the present disclosure is a semiconductor device and method of forming the same to improve the performance of FinFET devices by using lithography methods including forming a novel auxiliary layer and a novel photoresist layer. In the disclosed embodiments, the novel auxiliary layer and the novel photoresist layer can form a strong chemical bond therebetween to increase adhesion to each other to improve a peeling window of the photoresist layer, thus improving lithography performance. The disclosed processes and structures can improve the performance of the FinFET device.

Some embodiments contemplate both n-type devices, such as n-type FinFETs, and p-type devices, such as p-type FinFETs, being manufactured during a manufacturing process. Hence, some embodiments contemplate the formation of complementary devices. Figures below may illustrate one device, but one of ordinary skill in the art will readily understand that multiple devices, some with a different device type, can be formed during processing. Some aspects of the formation of complementary devices are discussed below, although such aspects may not necessarily be illustrated in the figures.

FIG. 11 illustrates an example of a FinFET in a three-dimensional view, in accordance with some embodiments. The FinFET comprises a fin 52 on a substrate 50 (e.g., a semiconductor substrate). Isolation regions 56 are disposed in the substrate 50, and the fin 52 protrudes above and from between neighboring isolation regions 56. Although the isolation regions 56 are described/illustrated as being separate from the substrate 50, as used herein the term “substrate” may be used to refer to just the semiconductor substrate or a semiconductor substrate inclusive of isolation regions. Additionally, although the fin 52 is illustrated as a single, continuous material as the substrate 50, the fin 52 and/or the substrate 50 may comprise a single material or a plurality of materials. In this context, the fin 52 refers to the portion extending between the neighboring isolation regions 56.

A gate dielectric layer 92 is along sidewalls and over a top surface of the fin 52, and a gate electrode 94 is over the gate dielectric layer 92. Source/drain regions 82 are disposed in opposite sides of the fin 52 with respect to the gate dielectric layer 92 and gate electrode 94. FIG. 1 further illustrates reference cross-sections that are used in later figures. Cross-section A-A is along a longitudinal axis of the gate electrode 94 and in a direction, for example, perpendicular to the direction of current flow between the source/drain regions 82 of the FinFET. Cross-section B-B is perpendicular to cross-section A-A and is along a longitudinal axis of the fin 52 and in a direction of, for example, a current flow between the source/drain regions 82 of the FinFET. Cross-section C-C is parallel to cross-section A-A and extends through a source/drain region of the FinFET. Subsequent figures refer to these reference cross-sections for clarity.

FIGS. 12A through 23C are cross-sectional views of intermediate stages in the manufacturing of FinFETs, in accordance with some embodiments. FIGS. 12A-16 illustrate reference cross-section A-A illustrated in FIG. 11, except for multiple fins/FinFETs. FIGS. 17A, 18A, 19A, 20A, 21A, 22A and 23A are illustrated along reference cross-section A-A illustrated in FIG. 11, and FIGS. 17B, 18B, 19B, 20B, 21B, 22B and 23B are illustrated along a similar cross-section B-B illustrated in FIG. 11, except for multiple gate structures. FIGS. 19C and 19D are illustrated along reference cross-section C-C illustrated in FIG. 11, except for multiple fins/FinFETs.

FIG. 12B is exemplary chemical bonds between a photoresist layer and an auxiliary layer in FIG. 12A in accordance with some embodiments. Reference is made to FIGS. 12A-12B. A substrate 50 is provided. The substrate may be similar to the substrate 111 in FIG. 2A, and thus the description thereof is omitted herein. The substrate 50 has a region 50N and a region 50P. The region 50N can be for forming n-type devices, such as NMOS transistors, e.g., n-type FinFETs. The region 50P can be for forming p-type devices, such as PMOS transistors, e.g., p-type FinFETs. The region 50N may be physically separated from the region 50P (as illustrated by divider 51), and any number of device features (e.g., other active devices, doped regions, isolation structures, etc.) may be disposed between the region 50N and the region 50P. In some embodiments, an auxiliary composition is applied on the substrate 50 to form an auxiliary layer 114, and a photoresist composition is applied on the auxiliary layer 114 to form the photoresist layer 116. Compositions and formation methods of the auxiliary layer 114 and the photoresist layer 116 are discussed previously with regard to FIGS. 2A-4B, and thus the description thereof is omitted herein.

The photoresist layer 116 can be patterned using the operations S108, S110, S112 of the method 1000 in FIG. 1. In FIG. 13, afterwards, the auxiliary layer 114 can be etched using the photoresist layer 116 as an etched mask using the operation similar to the operation S114 in the method 1000 in FIG. 1. As discussed previously with regard to FIGS. 4A-4B, since the photoresist layer 116 and the auxiliary layer 114 form the strong chemical bond therebetween to increase adhesion to each other, collapse or peeling of the photoresist layer 116 can be prevented. Peeling window of the photoresist layer 116 can be improved, thus improving lithography performance.

In FIG. 14, fins 52 are formed in the substrate 50. The fins 52 are semiconductor strips. In some embodiments, the fins 52 may be formed in the substrate 50 by etching trenches in the substrate 50. The etching may be any acceptable etch process, such as a reactive ion etch (RIE), neutral beam etch (NBE), the like, or a combination thereof. The etch may be anisotropic.

The fins may be patterned by any suitable method. For example, the fins may be patterned using one or more photolithography processes, including double-patterning or multi-patterning processes. Generally, double-patterning or multi-patterning processes combine photolithography and self-aligned processes, allowing patterns to be created that have, for example, pitches smaller than what is otherwise obtainable using a single, direct photolithography process. For example, in one embodiment, a sacrificial layer is formed over a substrate and patterned using a photolithography process. Spacers are formed alongside the patterned sacrificial layer using a self-aligned process. The sacrificial layer is then removed, and the remaining spacers may then be used to pattern the fins. In some embodiments, the mask (or other layer) may remain on the fins 52.

In FIG. 15A, an insulation material 54 is formed over the substrate 50 and between neighboring fins 52. The insulation material 54 may be an oxide, such as silicon oxide, a nitride, the like, or a combination thereof, and may be formed by a high density plasma chemical vapor deposition (HDP-CVD), a flowable CVD (FCVD) (e.g., a CVD-based material deposition in a remote plasma system and post curing to make it convert to another material, such as an oxide), the like, or a combination thereof. Other insulation materials formed by any acceptable process may be used. In the illustrated embodiment, the insulation material 54 is silicon oxide formed by a FCVD process. An anneal process may be performed once the insulation material is formed. In an embodiment, the insulation material 54 is formed such that excess insulation material 54 covers the fins 52. Although the insulation material 54 is illustrated as a single layer, some embodiments may utilize multiple layers. For example, in some embodiments a liner (not shown) may first be formed along a surface of the substrate 50 and the fins 52. Thereafter, a fill material, such as those discussed above may be formed over the liner.

A removal process is applied to the insulation material 54 to remove excess insulation material 54 over the fins 52. In some embodiments, a planarization process such as a chemical mechanical polish (CMP), an etch-back process, combinations thereof, or the like may be utilized. The planarization process exposes the fins 52 such that top surfaces of the fins 52 and the insulation material 54 are level after the planarization process is complete. In embodiments in which a mask remains on the fins 52, the planarization process may expose the mask or remove the mask such that top surfaces of the mask or the fins 52, respectively, and the insulation material 54 are level after the planarization process is complete.

In FIG. 15B, the insulation material 54 is recessed to form Shallow Trench Isolation (STI) regions 56. The insulation material 54 is recessed such that upper portions of fins 52 in the region 50N and in the region 50P protrude from between neighboring STI regions 56. Further, the top surfaces of the STI regions 56 may have a flat surface as illustrated, a convex surface, a concave surface (such as dishing), or a combination thereof. The top surfaces of the STI regions 56 may be formed flat, convex, and/or concave by an appropriate etch. The STI regions 56 may be recessed using an acceptable etching process, such as one that is selective to the material of the insulation material 54 (e.g., etches the material of the insulation material 54 at a faster rate than the material of the fins 52). For example, an oxide removal using, for example, dilute hydrofluoric (dHF) acid may be used.

Further in FIG. 15B, appropriate wells (not shown) may be formed in the fins 52 and/or the substrate 50. In some embodiments, a P well may be formed in the region 50N, and an N well may be formed in the region 50P. In some embodiments, a P well or an N well are formed in both the region 50N and the region 50P.

In the embodiments with different well types, the different implant steps for the region 50N and the region 50P may be achieved using a photoresist or other masks (not shown). For example, a photoresist may be formed over the fins 52 and the STI regions 56 in the region 50N. The photoresist is patterned to expose the region 50P of the substrate 50, such as a PMOS region. The photoresist can be formed by using a spin-on technique and can be patterned using acceptable photolithography techniques. Once the photoresist is patterned, an n-type impurity implant is performed in the region 50P, and the photoresist may act as a mask to substantially prevent n-type impurities from being implanted into the region 50N, such as an NMOS region. The n-type impurities may be phosphorus, arsenic, antimony, or the like. After the implant, the photoresist is removed, such as by an acceptable ashing process.

Following the implanting of the region 50P, a photoresist is formed over the fins 52 and the STI regions 56 in the region 50P. The photoresist is patterned to expose the region 50N of the substrate 50, such as the NMOS region. The photoresist can be formed by using a spin-on technique and can be patterned using acceptable photolithography techniques. Once the photoresist is patterned, a p-type impurity implant may be performed in the region 50N, and the photoresist may act as a mask to substantially prevent p-type impurities from being implanted into the region 50P, such as the PMOS region. The p-type impurities may be boron, boron fluoride, indium, or the like. After the implant, the photoresist may be removed, such as by an acceptable ashing process.

After the implants of the region 50N and the region 50P, an anneal may be performed to repair implant damage and to activate the p-type and/or n-type impurities that were implanted. In some embodiments, the grown materials of epitaxial fins may be in situ doped during growth, which may obviate the implantations, although in situ and implantation doping may be used together.

In FIG. 16, a dummy dielectric layer 60 is formed on the fins 52. The dummy dielectric layer 60 may be, for example, silicon oxide, silicon nitride, a combination thereof, or the like, and may be deposited or thermally grown according to acceptable techniques. A dummy gate layer 62 is formed over the dummy dielectric layer 60, and a mask layer 64 is formed over the dummy gate layer 62. The dummy gate layer 62 may be deposited over the dummy dielectric layer 60 and then planarized, such as by a CMP. The mask layer 64 may be deposited over the dummy gate layer 62. The dummy gate layer 62 may be a conductive or non-conductive material and may be selected from a group including amorphous silicon, polycrystalline-silicon (polysilicon), poly-crystalline silicon-germanium (poly-SiGe), metallic nitrides, metallic silicides, metallic oxides, and metals. The dummy gate layer 62 may be deposited by physical vapor deposition (PVD), CVD, sputter deposition, or other techniques known and used in the art for depositing the selected material. The dummy gate layer 62 may be made of other materials that have a high etching selectivity from the etching of isolation regions. The mask layer 64 may include, for example, silicon nitride, silicon oxynitride, or the like. In this example, a single dummy gate layer 62 and a single mask layer 64 are formed across the region 50N and the region 50P. It is noted that the dummy dielectric layer 60 is shown covering only the fins 52 for illustrative purposes only. In some embodiments, the dummy dielectric layer 60 may be deposited such that the dummy dielectric layer 60 covers the STI regions 56, extending between the dummy gate layer 62 and the STI regions 56.

FIGS. 17A through 23C illustrate various additional steps in the manufacturing of embodiment devices. FIGS. 17A through 23C illustrate features in either of the region 50N and the region 50P. For example, the structures illustrated in FIGS. 17A through 23C may be applicable to both the region 50N and the region 50P. Differences (if any) in the structures of the region 50N and the region 50P are described in the text accompanying each figure.

In FIGS. 17A and 17B, the mask layer 64 (see FIG. 16) may be patterned using acceptable photolithography and etching techniques to form masks 74. The pattern of the masks 74 then may be transferred to the dummy gate layer 62. In some embodiments (not illustrated), the pattern of the masks 74 may also be transferred to the dummy dielectric layer 60 by an acceptable etching technique to form dummy gates 72. The dummy gates 72 cover respective channel regions 58 of the fins 52. The pattern of the masks 74 may be used to physically separate each of the dummy gates 72 from adjacent dummy gates. The dummy gates 72 may also have a lengthwise direction substantially perpendicular to the lengthwise direction of respective fins 52.

Further in FIGS. 17A and 17B, gate seal spacers 80 can be formed on exposed surfaces of the dummy gates 72, the masks 74, and/or the fins 52. A thermal oxidation or a deposition followed by an anisotropic etch may form the gate seal spacers 80. The gate seal spacers 80 may be formed of silicon oxide, silicon nitride, silicon oxynitride, or the like.

After the formation of the gate seal spacers 80, implants for lightly doped source/drain (LDD) regions (not explicitly illustrated) may be performed. In the embodiments with different device types, similar to the implants discussed above in FIG. 15B, a mask, such as a photoresist, may be formed over the region 50N, while exposing the region 50P, and appropriate type (e.g., p-type) impurities may be implanted into the exposed fins 52 in the region 50P. The mask may then be removed. Subsequently, a mask, such as a photoresist, may be formed over the region 50P while exposing the region 50N, and appropriate type impurities (e.g., n-type) may be implanted into the exposed fins 52 in the region 50N. The mask may then be removed. The n-type impurities may be the any of the n-type impurities previously discussed, and the p-type impurities may be the any of the p-type impurities previously discussed. An anneal may be used to repair implant damage and to activate the implanted impurities.

In FIGS. 18A and 18B, gate spacers 86 are formed on the gate seal spacers 80 along sidewalls of the dummy gates 72 and the masks 74. The gate spacers 86 may be formed by conformally depositing an insulating material and subsequently anisotropically etching the insulating material. The insulating material of the gate spacers 86 may be silicon oxide, silicon nitride, silicon oxynitride, silicon carbonitride, a combination thereof, or the like.

It is noted that the above disclosure generally describes a process of forming spacers and LDD regions. Other processes and sequences may be used. For example, fewer or additional spacers may be utilized, different sequence of steps may be utilized (e.g., the gate seal spacers 80 may not be etched prior to forming the gate spacers 86, yielding “L-shaped” gate seal spacers, spacers may be formed and removed, and/or the like.) Furthermore, the n-type and p-type devices may be formed using a different structures and steps. For example, LDD regions for n-type devices may be formed prior to forming the gate seal spacers 80 while the LDD regions for p-type devices may be formed after forming the gate seal spacers 80.

In FIGS. 19A-19D, source/drain regions 82 are formed in the fins 52 to exert stress in the respective channel regions 58, thereby improving performance. The source/drain regions 82 are formed in the fins 52 such that each dummy gate 72 is disposed between respective neighboring pairs of the source/drain regions 82. In some embodiments the source/drain regions 82 may extend into, and may also penetrate through, the fins 52. In some embodiments, the gate spacers 86 are used to separate the source/drain regions 82 from the dummy gates 72 by an appropriate lateral distance so that the source/drain regions 82 do not short out subsequently formed gates of the resulting FinFETs.

The formation of the source/drain regions 82 may be formed by distinct processes, such that the source/drain regions 82 may be different materials in each region and may be formed by distinct processes. Various masking steps may be used to mask and expose appropriate regions when using distinct processes.

Referring first to FIGS. 18A and 18B, a patterning process is performed on the fins 52 to form recesses 85 in source/drain regions of the fins 52. The patterning process may be performed in a manner that the recesses 85 are formed between neighboring dummy gate stacks 72/74 (in interior regions of the fins 52), or between an isolation region 56 and adjacent dummy gate stacks 72/74 (in end regions of the fins 52). In some embodiments, the patterning process may include a suitable anisotropic dry etching process, while using the dummy gate stacks 72/74, the gate spacers 86, and/or isolation regions 56 as a combined mask. The suitable anisotropic dry etching process may include a reactive ion etch (RIE), neutral beam etch (NBE), the like, or a combination thereof. In some embodiments where the RIE is used in the first patterning process, process parameters such as, for example, a process gas mixture, a voltage bias, and an RF power may be chosen such that etching is predominantly performed using physical etching, such as ion bombardment, rather than chemical etching, such as radical etching through chemical reactions. In some embodiments, a voltage bias may be increased to increase energy of ions used in the ion bombardment process and, thus, increase a rate of physical etching. Since the physical etching is anisotropic in nature and the chemical etching is isotropic in nature, such an etching process has an etch rate in the vertical direction that is greater than an etch rate in the lateral direction.

In FIGS. 19A-19D, an epitaxial layer 82B of the source/drain regions 82 is grown in the recesses 85. In the region 50N, e.g., the NMOS region, the epitaxial layer 82B may include any acceptable material, such as appropriate for n-type FinFETs. For example, if the fin 52 is silicon, the epitaxial layer 82B in the region 50N may include materials exerting a tensile strain in the channel region 58, such as silicon, silicon carbide, phosphorous doped silicon carbide, silicon phosphide, or the like. The epitaxial layer 82B in the region 50N may have surfaces raised from respective surfaces of the fins 52 and may have facets. In some embodiments, the layers 82A and 82B are epitaxially grown in a single continuous process where the gas flows of the various precursors are changed to form the different layers. In some embodiments, the layers 82A and 82B are formed in separate, distinct epitaxial growth processes.

In the region 50P, e.g., the PMOS region, the epitaxial layer 82B may include any acceptable material, such as appropriate for p-type FinFETs. For example, if the fin 52 is silicon, the epitaxial layer 82B in the region 50P may comprise materials exerting a compressive strain in the channel region 58, such as silicon-germanium, boron doped silicon-germanium, germanium, germanium tin, or the like. The epitaxial layer 82B in the region 50P may also have surfaces raised from respective surfaces of the fins 52 and may have facets.

As a result of the epitaxy processes used to form the source/drain regions 82 in the region 50N and the region 50P, upper surfaces of the source/drain regions 82 have facets which expand laterally outward beyond sidewalls of the fins 52. In some embodiments, these facets cause adjacent epitaxial layers 82B of the source/drain regions 82 of a same FinFET to merge as illustrated by FIG. 19C. In other embodiments, adjacent epitaxial layers 82B of the source/drain regions 82 remain separated after the epitaxy process is completed as illustrated by FIG. 19D. In the embodiments illustrated in FIGS. 19C and 19D, gate spacers 86 are formed covering a portion of the sidewalls of the fins 52 that extend above the STI regions 56 thereby blocking the epitaxial growth. In some other embodiments, the spacer etch used to form the gate spacers 86 may be adjusted to remove the spacer material to allow the epitaxially grown region to extend to the surface of the STI region 56.

In FIGS. 20A and 20B, a first interlayer dielectric (ILD) 88 is deposited over the structure illustrated in FIGS. 19A and 19B. The first ILD 88 may be formed of a dielectric material, and may be deposited by any suitable method, such as CVD, plasma-enhanced CVD (PECVD), or FCVD. Dielectric materials may include phospho-silicate glass (PSG), boro-silicate glass (BSG), boron-doped phospho-silicate glass (BPSG), undoped silicate glass (USG), or the like. Other insulation materials formed by any acceptable process may be used. In some embodiments, a contact etch stop layer (CESL) 87 is disposed between the first ILD 88 and the epitaxial layer 82B of the source/drain regions 82, the masks 74, and the gate spacers 86. The CESL 87 may comprise a dielectric material, such as, silicon nitride, silicon oxide, silicon oxynitride, or the like, having a different etch rate than the material of the overlying first ILD 88.

In FIGS. 21A and 21B, a planarization process, such as a CMP, may be performed to level the top surface of the first ILD 88 with the top surfaces of the dummy gates 72 or the masks 74. The planarization process may also remove the masks 74 on the dummy gates 72, and portions of the gate seal spacers 80 and the gate spacers 86 along sidewalls of the masks 74. After the planarization process, top surfaces of the dummy gates 72, the gate seal spacers 80, the gate spacers 86, and the first ILD 88 are level. Accordingly, the top surfaces of the dummy gates 72 are exposed through the first ILD 88. In some embodiments, the masks 74 may remain, in which case the planarization process levels the top surface of the first ILD 88 with the top surfaces of the top surface of the masks 74.

In FIGS. 22A and 22B, the dummy gates 72, and the masks 74 if present, are removed in an etching step(s), so that recesses 90 are formed. Portions of the dummy dielectric layer 60 in the recesses 90 may also be removed. In some embodiments, only the dummy gates 72 are removed and the dummy dielectric layer 60 remains and is exposed by the recesses 90. In some embodiments, the dummy dielectric layer 60 is removed from recesses 90 in a first region of a die (e.g., a core logic region) and remains in recesses 90 in a second region of the die (e.g., an input/output region). In some embodiments, the dummy gates 72 are removed by an anisotropic dry etch process. For example, the etching process may include a dry etch process using reaction gas(es) that selectively etch the dummy gates 72 without etching the first ILD 88 or the gate spacers 86. Each recess 90 exposes and/or overlies a channel region 58 of a respective fin 52. Each channel region 58 is disposed between neighboring pairs of the source/drain regions 82. During the removal, the dummy dielectric layer 60 may be used as an etch stop layer when the dummy gates 72 are etched. The dummy dielectric layer 60 may then be optionally removed after the removal of the dummy gates 72.

In FIGS. 23A and 23B, gate dielectric layers 92 and gate electrodes 94 are formed for replacement gates. FIG. 23C illustrates a detailed view of region 89 of FIG. 23B. Gate dielectric layers 92 are deposited conformally in the recesses 90, such as on the top surfaces and the sidewalls of the fins 52 and on sidewalls of the gate seal spacers 80/gate spacers 86. The gate dielectric layers 92 may also be formed on the top surface of the first ILD 88. In accordance with some embodiments, the gate dielectric layers 92 comprise silicon oxide, silicon nitride, or multilayers thereof. In some embodiments, the gate dielectric layers 92 include a high-k dielectric material, and in these embodiments, the gate dielectric layers 92 may have a k value greater than about 7.0, and may include a metal oxide or a silicate of hafnium, aluminum, zirconium, lanthanum, manganese, barium, titanium, lead, and combinations thereof. The formation methods of the gate dielectric layers 92 may include molecular-beam deposition (MBD), ALD, PECVD, or the like. In embodiments where portions of the dummy dielectric layer 60 remains in the recesses 90, the gate dielectric layers 92 include a material of the dummy dielectric layer 60 (e.g., silicon oxide).

The gate electrodes 94 are deposited over the gate dielectric layers 92, respectively, and fill the remaining portions of the recesses 90. The gate electrodes 94 may include a metal-containing material such as titanium nitride, titanium oxide, tantalum nitride, tantalum carbide, cobalt, ruthenium, aluminum, tungsten, combinations thereof, or multi-layers thereof. For example, although a single layer gate electrode 94 is illustrated in FIG. 23B, the gate electrode 94 may comprise any number of liner layers 94A, any number of work function tuning layers 94B, and a fill material 94C as illustrated by FIG. 23C. After the filling of the recesses 90, a planarization process, such as a CMP, may be performed to remove the excess portions of the gate dielectric layers 92 and the material of the gate electrodes 94, which excess portions are over the top surface of the first ILD 88. The remaining portions of material of the gate electrodes 94 and the gate dielectric layers 92 thus form replacement gates of the resulting FinFETs. The gate electrodes 94 and the gate dielectric layers 92 may be collectively referred to as a “gate stack.” The gate and the gate stacks may extend along sidewalls of a channel region 58 of the fins 52.

The formation of the gate dielectric layers 92 in the region 50N and the region 50P may occur simultaneously such that the gate dielectric layers 92 in each region are formed from the same materials, and the formation of the gate electrodes 94 may occur simultaneously such that the gate electrodes 94 in each region are formed from the same materials. In some embodiments, the gate dielectric layers 92 in each region may be formed by distinct processes, such that the gate dielectric layers 92 may be different materials, and/or the gate electrodes 94 in each region may be formed by distinct processes, such that the gate electrodes 94 may be different materials. Various masking steps may be used to mask and expose appropriate regions when using distinct processes.

Embodiments may achieve advantages. One advantage is that the novel photoresist layer and the novel auxiliary layer could form a strong chemical bond therebetween to increase adhesion to each other. Therefore, collapse or peeling of the photoresist layer can be prevented. Peeling window of the photoresist layer can be improved, thus improving lithography performance.

In some embodiments, a lithography method includes the following steps. An auxiliary composition is applied over a substrate to form an auxiliary layer, wherein the auxiliary composition comprises a first polymer, the first polymer comprises a first functional group including a first boronic acid (BOH₂) group, a first multi-OH group, or a combination thereof. A photoresist composition is applied over the auxiliary layer to form a photoresist layer. The photoresist layer is exposed. The photoresist layer is developed. In some embodiments, one of the first boronic acid (BOH₂) group and the first multi-OH group is a t-butyl acetate group, an O-nitrobenzyl group or a pyrene group. In some embodiments, one of the first boronic acid (BOH₂) group and the first multi-OH group has a formula (a3), formula (a4) or formula (a5):

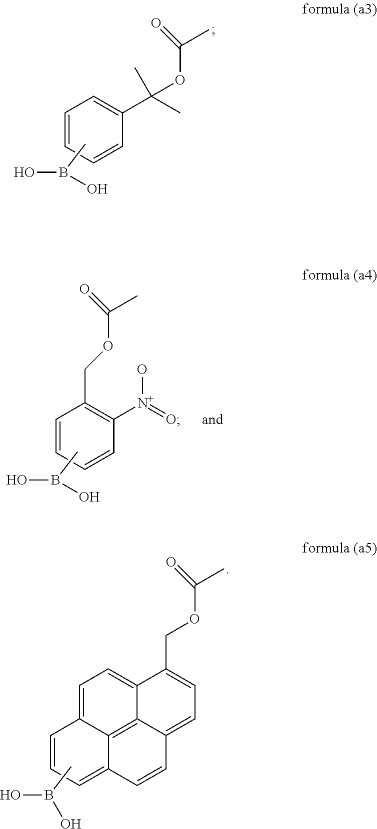

In some embodiments, the first multi-OH group has a formula (b1)-(b5):

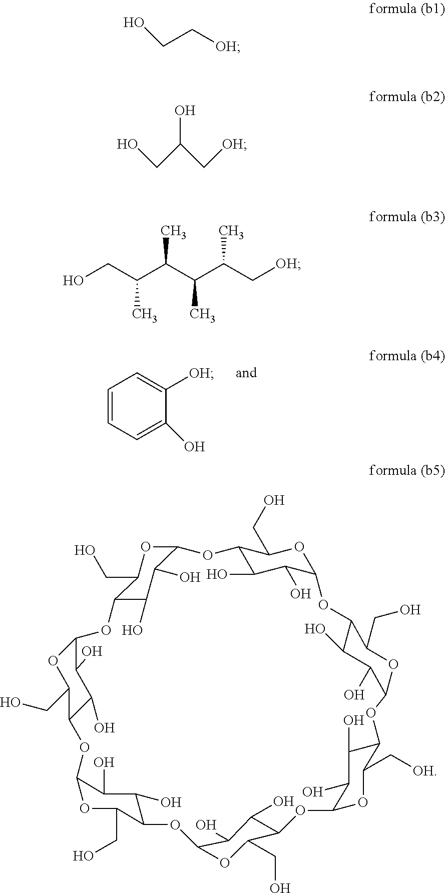

In some embodiments, the lithography method further comprises after applying the photoresist composition over the auxiliary layer to form the photoresist layer, performing a bake operation to the photoresist layer and the auxiliary layer to crosslink the photoresist layer and the auxiliary layer. In some embodiments, the photoresist composition comprises a second polymer, the second polymer comprises a second functional group including a second boronic acid (BOH₂) group, a second multi-OH group, or a combination thereof. In some embodiments, the second functional group is a t-butyl acetate group, an O-nitrobenzyl group or a pyrene group. In some embodiments, one of the second boronic acid (BOH₂) group and the second multi-OH group has a formula (a3), formula (a4) or formula (a5):

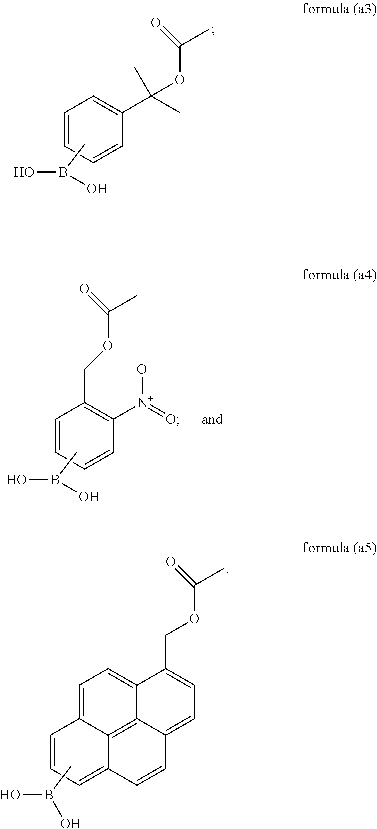

In some embodiments, the second multi-OH group has a formula (b1)-(b5):

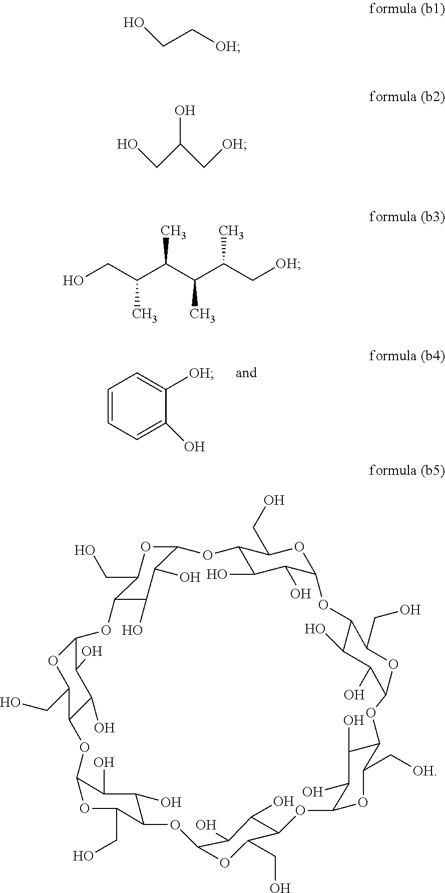

In some embodiments, a multilayer stack comprises an auxiliary layer and a photoresist layer over the auxiliary layer. The auxiliary layer comprises a first polymer, the first polymer comprises a first functional group including a first boronic acid (BOH₂) group, a first multi-OH group, or a combination thereof. In some embodiments, one of the first boronic acid (BOH₂) group and the first multi-OH group is a t-butyl acetate group, an O-nitrobenzyl group or a pyrene group. In some embodiments, one of the first boronic acid (BOH₂) group and the first multi-OH group has a formula (a3), formula (a4) or formula (a5):

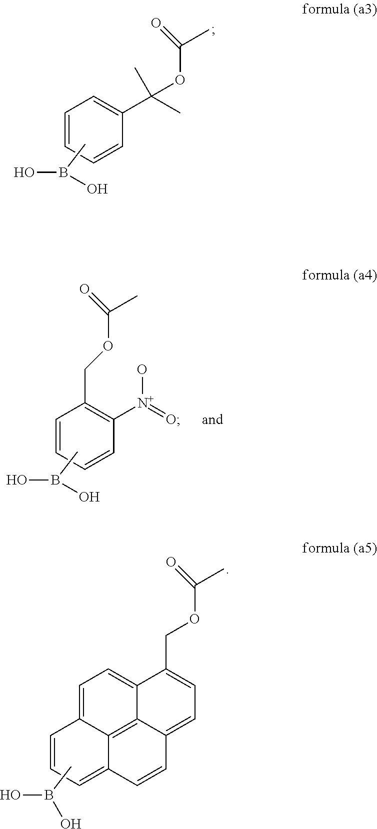

In some embodiments, in the first multi-OH group has a formula (b1)-(b5):

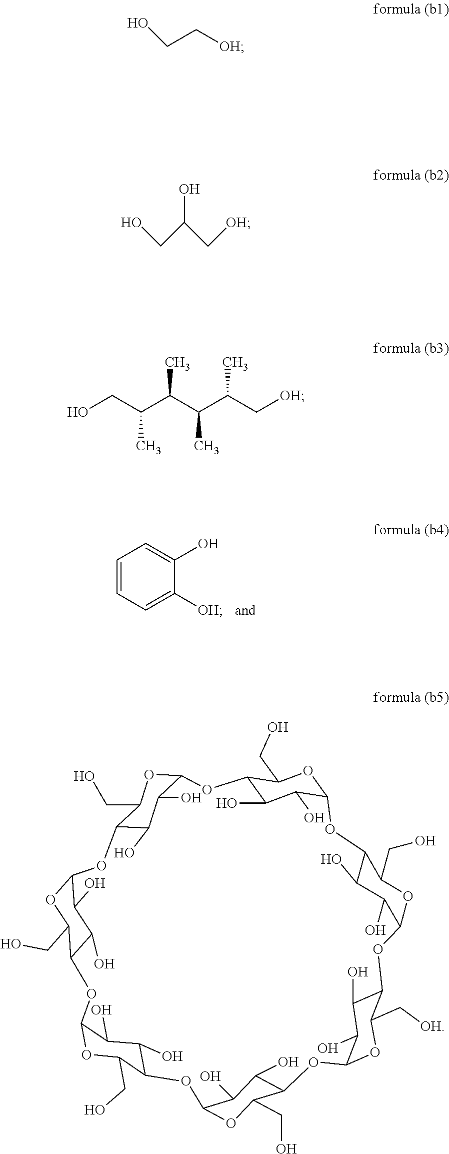

In some embodiments, the photoresist layer comprises a second polymer, the second polymer comprises a second functional group including a second boronic acid (BOH₂) group, a second multi-OH group, or a combination thereof. In some embodiments, one of the second boronic acid (BOH₂) group and the second multi-OH group is a t-butyl acetate group, an O-nitrobenzyl group or a pyrene group. In some embodiments, one of the second boronic acid (BOH₂) group and the second multi-OH group has a formula (a3), formula (a4) or formula (a5):

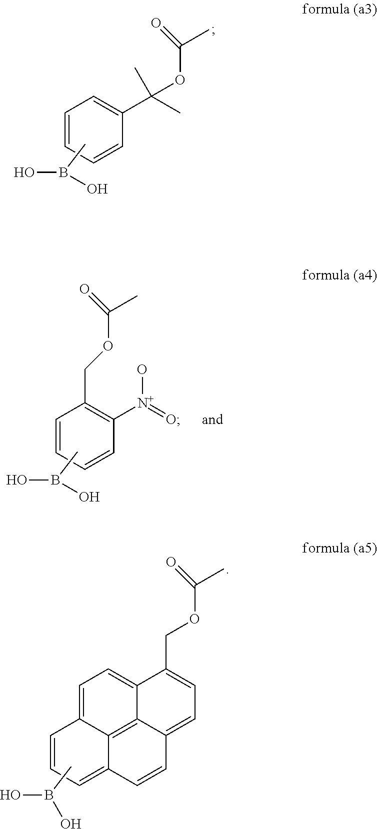

In some embodiments, the second multi-OH group has a formula (b1)-(b5):

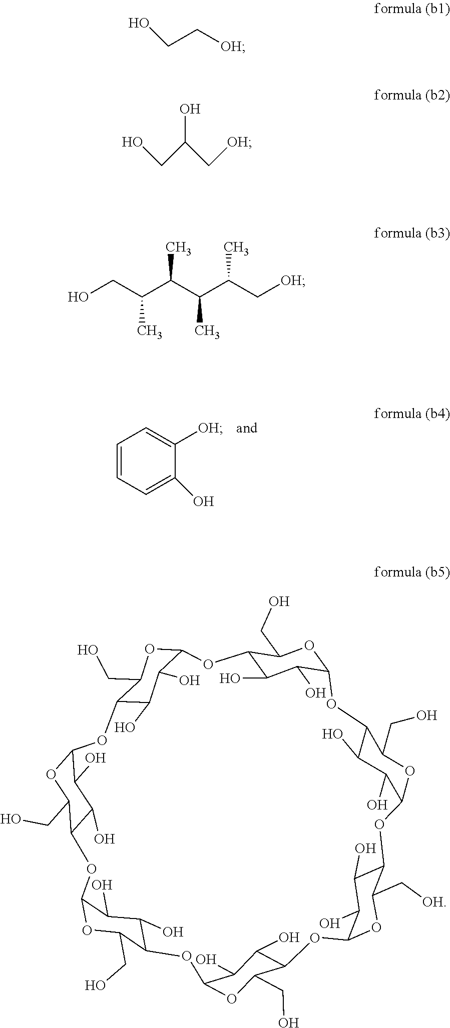

In some embodiments, a photoresist composition comprises a polymer, a photoacid generator and a solvent. The polymer comprises a functional group including a second boronic acid (BOH₂) group, a second multi-OH group, or a combination thereof. In some embodiments, one of the second boronic acid (BOH₂) group and the second multi-OH group is a t-butyl acetate group, an O-nitrobenzyl group or a pyrene group. In some embodiments, one of the second boronic acid (BOH₂) group and the second multi-OH group has a formula (a3), formula (a4) or formula (a5):

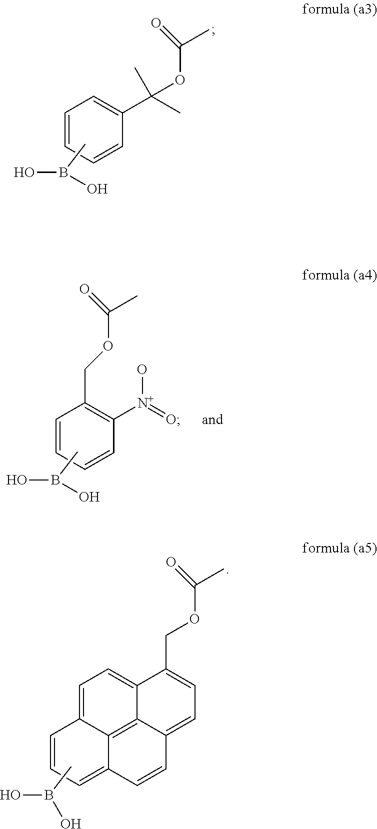

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A lithography method, comprising:
applying an auxiliary composition over a substrate to form an auxiliary layer, wherein the auxiliary composition comprises a first polymer, the first polymer comprises a first functional group including a first boronic acid (BOH₂) group, a first multi-OH group, or a combination thereof;
applying a photoresist composition over the auxiliary layer to form a photoresist layer;
exposing the photoresist layer; and
developing the photoresist layer.

2. The lithography method of claim 1, wherein one of the first boronic acid (BOH₂) group and the first multi-OH group is a t-butyl acetate group, an O-nitrobenzyl group or a pyrene group.

3. The lithography method of claim 1, wherein one of the first boronic acid (BOH₂) group and the first multi-OH group has a formula (a3), formula (a4) or formula (a5):

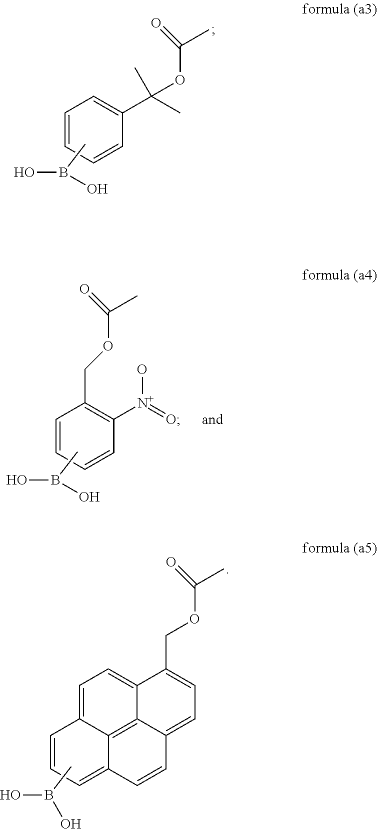

4. The lithography method of claim 1, wherein the first multi-OH group has a formula (b1)-(b5):

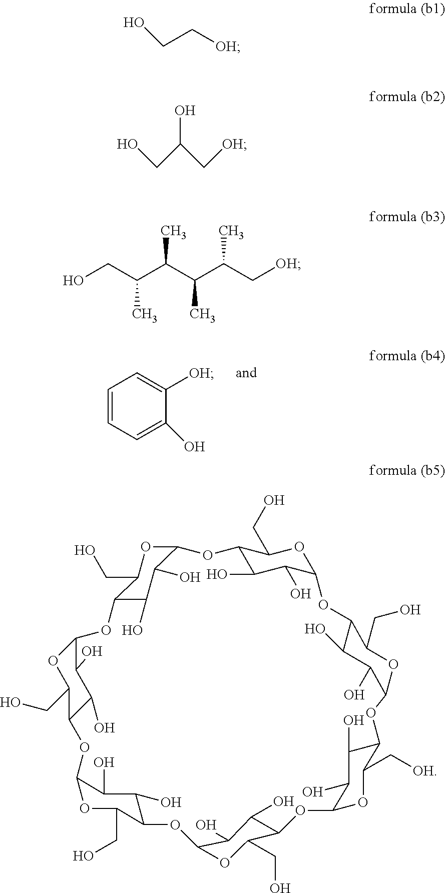

5. The lithography method of claim 1, further comprising:
after applying the photoresist composition over the auxiliary layer to form the photoresist layer, performing a bake operation to the photoresist layer and the auxiliary layer to crosslink the photoresist layer and the auxiliary layer.

6. The lithography method of claim 1, wherein the photoresist composition comprises a second polymer, the second polymer comprises a second functional group including a second boronic acid (BOH₂) group, a second multi-OH group, or a combination thereof.

7. The lithography method of claim 6, wherein the second functional group is a t-butyl acetate group, an O-nitrobenzyl group or a pyrene group.

8. The lithography method of claim 6, wherein one of the second boronic acid (BOH₂) group and the second multi-OH group has a formula (a3), formula (a4) or formula (a5):

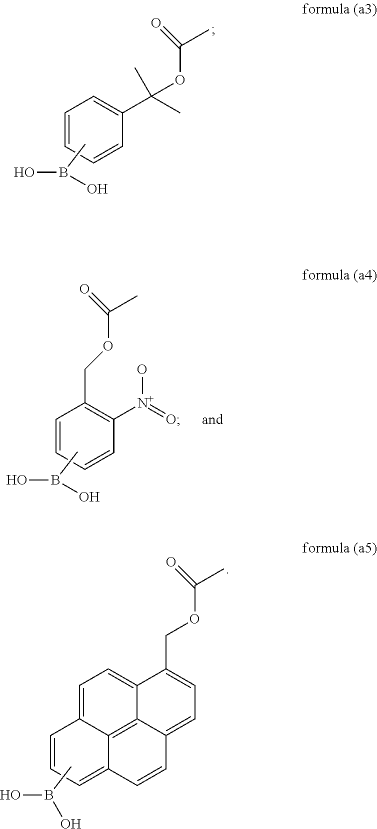

9. The lithography method of claim 6, wherein the second multi-OH group has a formula (b1)-(b5):

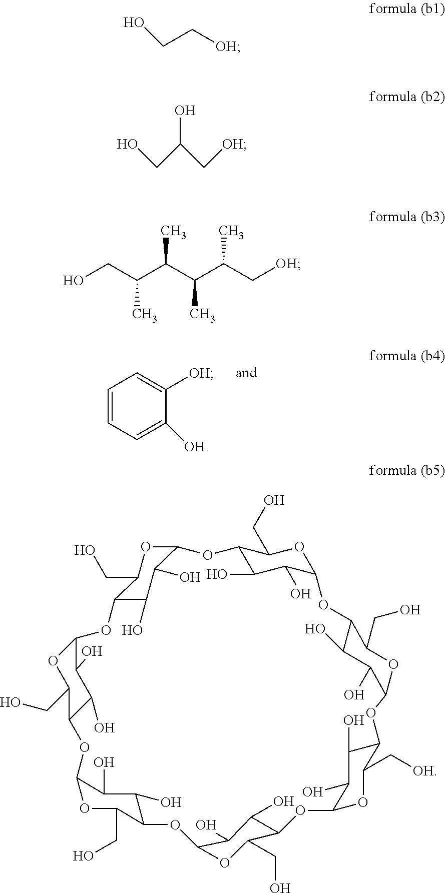

10. A multilayer stack, comprising:
an auxiliary layer, wherein the auxiliary layer comprises a first polymer, the first polymer comprises a first functional group including a first boronic acid (BOH₂) group, a first multi-OH group, or a combination thereof; and
a photoresist layer over the auxiliary layer.

11. The multilayer stack of claim 10, wherein one of the first boronic acid (BOH₂) group and the first multi-OH group is a t-butyl acetate group, an O-nitrobenzyl group or a pyrene group.

12. The multilayer stack of claim 10, wherein one of the first boronic acid (BOH₂) group and the first multi-OH group has a formula (a3), formula (a4) or formula (a5):

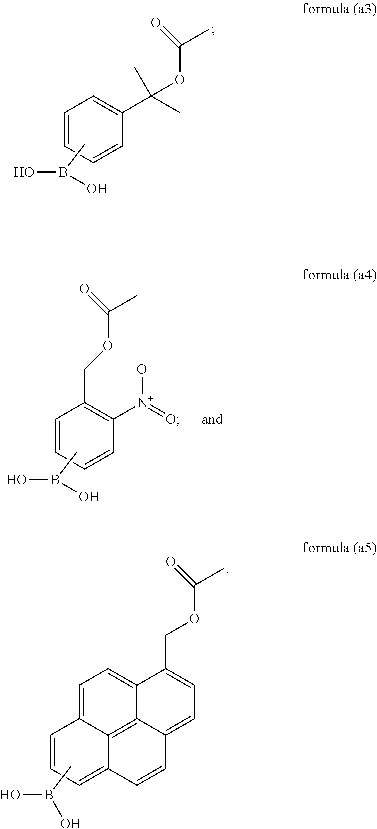

13. The multilayer stack of claim 10, wherein the first multi-OH group has a formula (b1)-(b5):

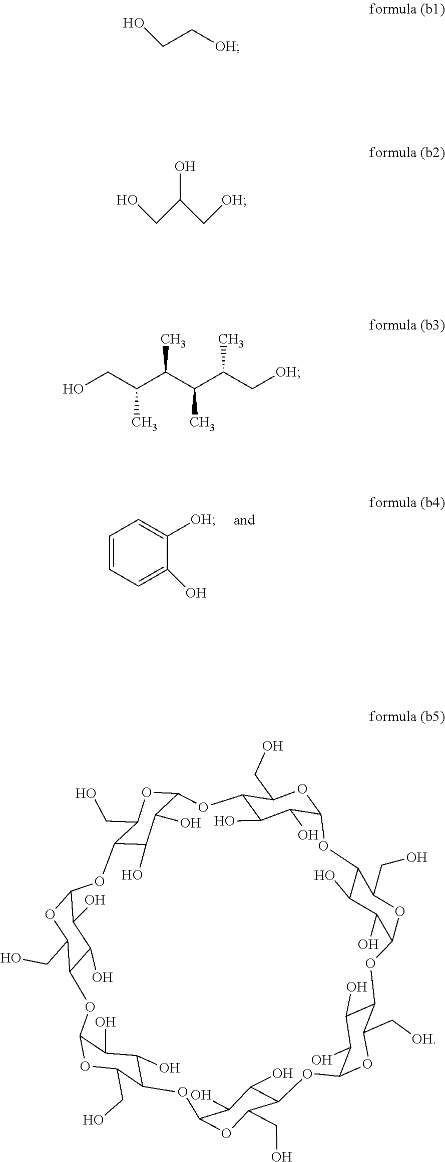

14. The multilayer stack of claim 10, wherein the photoresist layer comprises a second polymer, the second polymer comprises a second functional group including a second boronic acid (BOH₂) group, a second multi-OH group, or a combination thereof.

15. The multilayer stack of claim 14, wherein one of the second boronic acid (BOH₂) group and the second multi-OH group is a t-butyl acetate group, an O-nitrobenzyl group or a pyrene group.

16. The multilayer stack of claim 14, wherein one of the second boronic acid (BOH₂) group and the second multi-OH group has a formula (a3), formula (a4) or formula (a5):

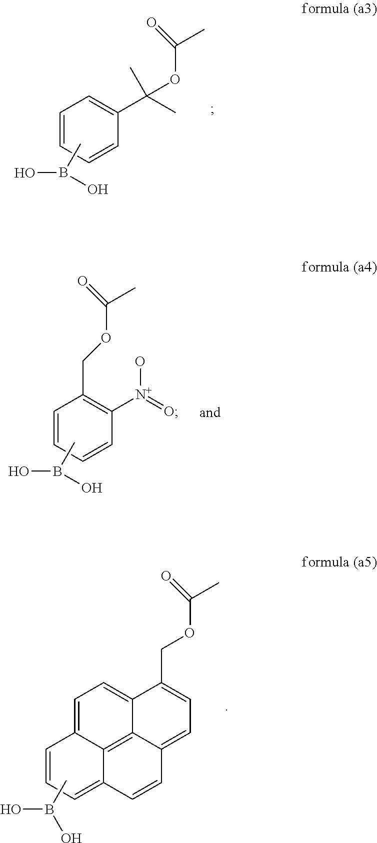

17. The multilayer stack of claim 14, wherein the second multi-OH group has a formula (b1)-(b5):

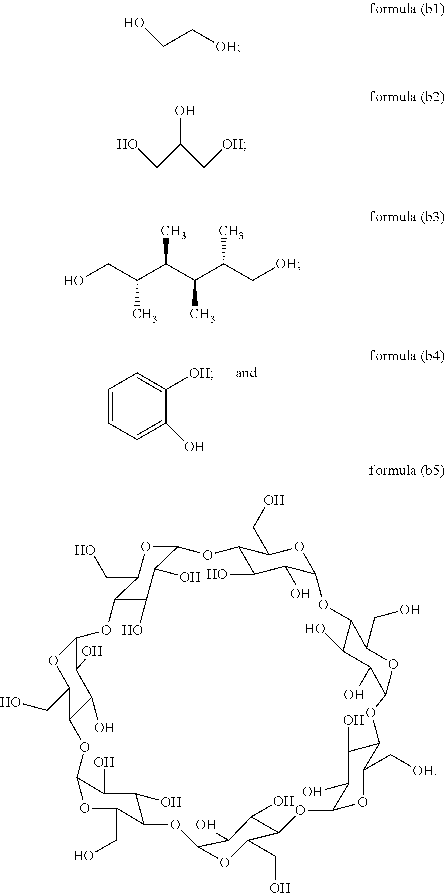

18. A photoresist composition, comprising:
a polymer, comprising a functional group including a second boronic acid (BOH₂) group, a second multi-OH group, or a combination thereof;
a photoacid generator; and
a solvent.

19. The photoresist composition of claim 18, wherein one of the second boronic acid (BOH₂) group and the second multi-OH group is a t-butyl acetate group, an O-nitrobenzyl group or a pyrene group.

20. The photoresist composition of claim 18, wherein one of the second boronic acid (BOH₂) group and the second multi-OH group has a formula (a3), formula (a4) or formula (a5): [chemical structure]
